(19) United States
(12) Patent Application Publication
(10) Pub. No.: US 2026/0263527 A1
(43) Pub. Date: Sep. 10, 2026

(54) APPARATUS FOR USE IN TISSUE REPAIR

(71) Applicants: The University of Melbourne, Victoria (AU); University of Wollongong, New South Wales (AU); St Vincent's Hospital (Melbourne) Limited, Victoria (AU)

(72) Inventors: Peter Fook Meng Choong, Victoria (AU); Serena Duchi, Victoria (AU); Carmine Onofrillo, Victoria (AU); Gordon George Wallace, New South Wales (AU); Stephen Thomas Beirne, New South Wales (AU); Sanjeev Gambhir, New South Wales (AU); Daniel John Gailer, New South Wales (AU); Claudia Di Bella, Victoria (AU)

(73) Assignees: The University of Melbourne, Victoria (AU); University of Wollongong, New South Wales (AU); St Vincent's Hospital (Melbourne) Limited, Victoria (AU)

(21) Appl. No.: 19/489,216

(22) PCT Filed: May 31, 2024
(86) PCT No.: PCT/AU2024/050581

§ 371 Date: Dec. 1, 2025

(30) Foreign Application Priority Data
May 31, 2023 (AU) 2023901716

(51) Int. Cl. A61K 35/35

(52) U.S. Cl. CPC A61K 35/35

(57) ABSTRACT

An apparatus for use in preparation of a composition for treatment or repair or regeneration of tissue comprises a collection vessel configured to receive harvested tissue and at least one filter element located inside the collection vessel. The apparatus is configured to be received in a centrifuge to separate a Stromal Vascular Fraction (SVF) from the harvested tissue within the collection vessel. At least a portion or extract of the SVF separated from the harvested tissue in the apparatus is used in preparation of a treatment composition comprising a mixture of SVF or extract and a polymer composition for treatment or repair or regeneration of the tissue.

### TECHNICAL FIELD

The present disclosure relates to preparation of a composition for repair or regeneration of tissue. It relates particularly, but not exclusively, to apparatuses, kits, systems and methods for preparation of a composition for repair or regeneration of cartilage, and treatment of a cartilage defect using said composition.

### BACKGROUND OF INVENTION

Articular (hyaline) cartilage is specialised tissue that lines the surface of long bones and is critical for normal joint function. Human cartilage has a poor ability to self-repair, meaning small defects after injury tend to enlarge or deteriorate over time. The degeneration of cartilage leads to osteoarthritis, a painful and debilitating condition which is a major contributor to the global burden of disease. Despite the prevalence and severity of osteoarthritis, there are few clinical interventions which are effective at preventing its onset, even when an early stage chondral defect has been detected.

Known interventions include: microfracture in which small holes are punched through to underlying bone beneath the defect site in order to recruit regenerative stem cells from the bone marrow, mosaicplasty in which healthy cartilage is harvested from non-load-bearing regions of the joint and used to inlay the defect area, autologous chondrocyte implantation (ACI) in which patient chondrocytes are expanded in the lab and then reimplanted without a matrix, and matrix-assisted autologous chondrocyte implantation (MACI) in which patient chondrocytes are expanded in the laboratory and then reimplanted within a collagen matrix. The end result of all of these interventions, however, is usually a defect filled with fibrocartilage. Fibrocartilage is less durable than the hyaline type cartilage, and although appearing to fill a defect soon after natural attempts at healing or following treatment, is not durable nor resilient to normal activity and soon degenerates again. This contrasts with hyaline cartilage which is the hallmark of healthy articular cartilage tissue. There is as yet no clinical intervention that results in long term restoration of functional hyaline cartilage.

It would be desirable to overcome or ameliorate one or more of the shortcomings of these existing interventions, or at least provide a useful alternative.

The discussion of the background to the invention included herein including reference to documents, acts, materials, devices, articles and the like is included to explain the context of the present disclosure. This is not to be taken as an admission or a suggestion that any of the material referred to was published, known or part of the common general knowledge as at the priority date of any of the provisional claims.

### SUMMARY OF INVENTION

Viewed from one aspect, the present disclosure provides an apparatus for preparation of a composition for treatment or repair or regeneration of tissue, the apparatus comprising: a collection vessel configured to receive harvested tissue; and at least one filter element located inside the collection vessel to separate a cellular fraction (e.g., a Stromal Vascular Fraction (SVF)) or a cellular extract from the harvested tissue within the collection vessel; wherein the apparatus is configured to: receive a volume of a polymer composition for mixing with the cellular fraction (e.g., SVF) or cellular extract obtained from the harvested tissue; and provide a prepared composition comprising a mixture of the cellular fraction (e.g., SVF) or cellular extract and the polymer composition for treatment or repair or regeneration of the tissue.

In some embodiments, the apparatus comprises a delivery vessel, couplable with the collection vessel, for containing the prepared composition which is to be delivered to the site of defect or injury.

In some embodiments, at least one of the collection vessel and the delivery vessel is pre-loaded with a volume of the polymer composition. In some embodiments, the pre-loaded polymer composition is in a solid or gel state and the pre-loaded collection vessel and/or delivery vessel is able to receive a solid to liquid phase change agent for liquefying the polymer. The solid to liquid phase change agent may comprise, for example, a thermal mechanism or a chemical agent.

In some embodiments, at least one of the collection vessel and the delivery vessel is configured to receive a volume of liquid polymer composition before mixing with the cellular fraction (e.g., SVF) or cellular extract.

In some embodiments, the at least one filter element comprises a plurality of openings of about 30 to 60 μm, preferably about 40 μm.

In some embodiments, the apparatus may comprise a valve for coupling between the collection vessel and a delivery vessel.

In some embodiments, the at least one filter element comprises at least one strainer attached to an interior wall of the collection vessel.

In some embodiments, the filter element comprises a hollow filter barrel configured to be received within the collection vessel, and to receive the harvested tissue therein, the filter barrel comprising at least one strainer configured for separation of the cellular fraction (e.g., SVF) or cellular extract from the received harvested tissue.

In some embodiments, the collection vessel is configured for use in a centrifuge. Alternatively/additionally, the collection vessel may be configured to be received in a container for use in a centrifuge.

In some embodiments, the delivery vessel is couplable with a delivery device.

In some embodiments, the polymer composition is functionalised to achieve in situ stabilisation of the polymer composition under activation conditions.

In some embodiments, the polymer composition comprises a photoinitiator and the activation conditions comprise exposure to light such as visible light comprising a wavelength of about 405 nm, optionally at about 20 mW/cm2. The activation conditions may comprise exposure to the light for about 60 to about 120 seconds.

In some embodiments, the polymer composition comprises at least one of: gelatin methacryloyl (GelMA) (e.g., Type A gelatin obtained from porcine skin, bloom 300, with from about 80% to about 90% methacryloyl functionalisation); and methacrylated alginate-RGD (e.g., about 270 to about 280 kDa alginate with M/G ratio of approx. 1.3-1.4, about 40-60% (preferably 40-50%, e.g., 45-46%) methacrylate functionalisation (as a % of functionalised monomers) and 5-6% RGD (as a wt % substitution of reactive groups).

In some embodiments of the apparatus, the harvested tissue is adipose tissue, preferably infrapatellar fat pad tissue.

In some embodiments of the apparatus, the tissue being treated or repaired or regenerated is cartilage, preferably articular cartilage.

An aspect of the present disclosure also provides a method for treating, or repairing, or regenerating tissue comprising use of the apparatus according to the foregoing.

An aspect of the present disclosure also provides a composition for treating, or repairing, or regenerating tissue, prepared using the apparatus or the method according to the foregoing.

Viewed from another aspect, the present disclosure provides a method for repairing or regenerating a tissue to treat a tissue defect comprising the steps of: processing harvested tissue to separate a cellular fraction (e.g., Stromal Vascular Fraction (SVF)) or cellular extract from the harvested tissue; preparing a cell loaded polymer composition by mixing the cellular fraction (e.g., SVF) or cellular extract with a polymer composition; administering the cell loaded polymer composition to the tissue defect; and activating the cell loaded polymer composition for in situ stabilisation of the polymer composition to allow for cell ingrowth and tissue repair or regeneration; wherein the method is performed in a single sterile environment.

In some embodiments, the method comprises the step of obtaining the harvested tissue from a donor subject in the single sterile environment.

In some embodiments, processing the harvested tissue comprises using at least one filter while centrifuging to separate the cellular fraction (e.g., SVF) or cellular extract from the remainder of the harvested tissue.

In some embodiments, the polymer composition is pre-loaded in at least one of a collection vessel into which the cellular fraction (e.g., SVF) or cellular extract is received, and a delivery vessel from which the cell loaded polymer composition is delivered to the site of defect or injury.

In some embodiments, the pre-loaded polymer composition is in a solid or gel state and the method comprises inducing a solid to liquid phase change before mixing the polymer composition with the cellular fraction (e.g., SVF) or cellular extract. In some embodiments, the solid to liquid phase change is achieved by at least one of a thermal mechanism and a chemical agent.

In some embodiments, at least one of a collection vessel and a delivery vessel is preloaded with a liquid volume of the polymer composition before mixing with the cellular fraction (e.g., SVF) or cellular extract.

In some embodiments, mixing comprises, at least, transferring the cellular fraction (e.g., SVF) or cellular extract and any polymer composition from the collection vessel into a delivery vessel where the cellular fraction (e.g., SVF) or cellular extract in mixture with the polymer composition, forms the cell loaded polymer composition.

In some embodiments, the activating step achieves in situ stabilisation of the polymer composition.

In some embodiments, the polymer composition contains a photoinitiator and the activating step comprises exposing the cell loaded polymer composition to light. In some embodiments, the method comprises adding a photoinitiator before administering the cell loaded polymer composition to the tissue defect.

In some embodiments, the activating step comprises exposing the cell loaded polymer composition containing a photoinitiator to visible light comprising a wavelength of about 405 nm at about 20 mW/cm² for about 60 toto about 120 seconds.

In some embodiments, the polymer composition comprises at least one of: gelatin methacryloyl (GelMA) (e.g., Type A gelatin obtained from porcine skin, bloom 300, with from about 80% to about 90% methacryloyl functionalisation); and methacrylated alginate-RGD (e.g., about 270 to about 280 kDa alginate with M/G ratio of approx. 1.3-1.4, about 40-60% (preferably 40-50%, e.g., 45-46%) methacrylate functionalisation and 5-6% RGD (as a wt % substitution or reactive groups).

In some embodiments of the method, the harvested tissue is adipose tissue, preferably infrapatellar fat pad tissue.

In some embodiments of the method, the tissue being treated or repaired or regenerated is cartilage, preferably articular cartilage.

An aspect of the present disclosure also provides the method according to the foregoing, performed using the apparatus according to the foregoing.

Another aspect of the present disclosure provides a kit for treatment or repair or regeneration of tissue, the kit comprising the apparatus according to the foregoing, provided in pre-assembled or unassembled condition in a kit container.

In some embodiments, the kit comprises a solid to liquid phase change agent being a thermal heat source (e.g., incubator) and/or a chemical agent (e.g., a chelator such as EDTA).

In some embodiments, the kit comprises a photoinitiator.

In some embodiments, the kit comprises a delivery device couplable with the delivery vessel.

In some embodiments, the kit comprises a pre-treatment component configured to mechanically process the harvested tissue before it is received in the collection vessel.

In some embodiments, the kit comprises a light source for activating the prepared composition in vivo.

Viewed from another aspect, the present disclosure provides an apparatus for use in preparation of a composition for treatment or repair or regeneration of tissue, the apparatus comprising: a collection vessel configured to receive harvested tissue; and at least one filter element located inside the collection vessel; wherein the apparatus is configured to be received in a centrifuge to separate a Stromal Vascular Fraction (SVF) from the harvested tissue within the collection vessel; wherein at least a portion or extract of the SVF separated from the harvested tissue in the apparatus is used in preparation of a treatment composition comprising a mixture of SVF or extract and a polymer composition for treatment or repair or regeneration of the tissue.

In some embodiments, the apparatus comprises a collection container for receiving the separated SVF and which is removably couplable with the collection vessel. The apparatus comprising the collection container when coupled with the collection vessel may be configured to be received within a centrifuge, or within a centrifuge tube, for use in a centrifuge.

The at least one filter element may comprise a plurality of openings of about 30 to 60 um, preferably about 40 μm. The openings may be provided in a strainer of the filter element a will be explained below.

In some embodiments, the filter element comprises a hollow filter barrel configured to be received within the collection vessel, and to receive the harvested tissue therein in sue. The filter barrel comprises at least one strainer configured for separation of the SVF from the received harvested tissue.

In some embodiments, the apparatus comprises a spacer to separate the filter barrel from an end of the collection vessel when in use.

In some embodiments, the apparatus comprises or is provided with a polymer container containing the polymer composition. The polymer container may comprise a syringe or other suitable container. In some embodiments the polymer container may comprise one or both of the collection vessel and a pre-loaded syringe. The polymer container may comprise a volume of liquid polymer composition. In some embodiments, and optionally wherein the volume of liquid polymer composition in the polymer container is about 0.5 mL to about 3 mL such as about 1 mL.

In some embodiments, the apparatus comprises or is provided with a delivery vessel for receiving the prepared treatment composition, and optionally wherein the delivery vessel is couplable with a delivery device.

In some embodiments, the polymer composition is functionalised to achieve in situ stabilisation of the polymer composition under activation conditions. The polymer composition may comprise a photoinitiator and the activation conditions may comprise exposure to visible light. The visible light may comprise a wavelength of about 405 nm, optionally at about 20 mW/cm² and optionally for about 60 seconds to about 150 seconds, preferably about 120 seconds.

In some embodiments, the polymer composition comprises at least one of: gelatin methacryloyl (GelMA); and methacrylated alginate-RGD.

In some embodiments, the harvested tissue is adipose tissue, preferably infrapatellar fat tissue.

In some embodiments, the tissue being treated or repaired is cartilage, preferably articular cartilage.

An aspect of the present disclosure also provides a method for treating, or repairing, or regenerating tissue comprising use of the apparatus according to the foregoing.

An aspect of the present disclosure also provides a composition for treating, or repairing, or regenerating tissue, prepared using the apparatus or the method according to the foregoing.

Viewed from another aspect, the present disclosure provides a method for repairing or regenerating tissue to treat a tissue defect comprising the steps of: processing harvested tissue to separate a Stromal Vascular Fraction (SVF) from the harvested tissue; preparing a treatment composition comprising a cell loaded polymer composition by mixing at least a portion of the SVF or an extract thereof with a polymer composition; administering the treatment composition comprising the cell loaded polymer composition to the tissue defect; and activating the cell loaded polymer composition for in situ stabilisation of the polymer composition to allow for cell ingrowth and tissue repair or regeneration; wherein the method is performed in a single sterile environment.

In some embodiments the method comprises the step of obtaining the harvested tissue from a donor subject in the single sterile environment. The donor subject may be a patient having the tissue defect to be treated.

In some embodiments processing the harvested tissue comprises using at least one filter while centrifuging to separate the SVF.

In some embodiments the method comprises collecting harvested tissue using an attachment connected in a suction path of an arthroscope instrument used to remove the harvested tissue from the donor subject.

In some embodiments mixing comprises, at least, transferring at least a portion the separated SVF or extract thereof and a volume of the polymer composition into a delivery vessel where the SVF portion or extract thereof, in mixture with the polymer composition, forms the cell loaded polymer composition. The treatment composition may comprise the cell loaded polymer composition alone or in combination with one or more bioactive molecules (for example, one or more of growth factors, interleukins, and anti-clotting agents) or other factors.

In some embodiments mixing comprises combining a volume of the separated SVF or extract thereof with a buffer solution before mixing with the polymer composition.

In some embodiments mixing comprises transferring a volume of the polymer composition into the combined SVF or extract and buffer solution, or vice versa, to form the cell loaded polymer composition.

In some embodiments the treatment composition comprising the cell loaded polymer composition contains a photoinitiator and the activating step comprises exposing the treatment composition comprising the cell loaded polymer composition to light.

In some embodiments the activating step comprises exposing the treatment composition comprising the cell loaded polymer composition to visible light. The visible light may comprise a wavelength of about 405 nm, optionally at about 20 mW/cm² and optionally for about 60 seconds to about 150 seconds, preferably about 120 seconds.

In some embodiments the polymer composition comprises at least one of: gelatin methacryloyl (GelMA); and methacrylated alginate-RGD.

In some embodiments the harvested tissue is adipose tissue, preferably infrapatellar fat tissue.

In some embodiments the tissue being treated or repaired is cartilage, preferably articular cartilage.

An aspect of the present disclosure also provides the method according to the foregoing, performed using the apparatus according to the foregoing.

Another aspect of the present disclosure provides a kit for preparation of a composition for treatment or repair or regeneration of tissue, the kit comprising: the apparatus according to the foregoing. Components of the kit may be provided in pre-assembled or unassembled condition in a kit container.

In some embodiments, the kit comprises an attachment for collecting harvested tissue, the attachment configured to be connected in a suction path of an arthroscope instrument used to remove the harvested tissue from a patient.

In some embodiments, the kit comprises a light source for activating the prepared composition in vivo.

Another aspect of the present disclosure provides an attachment for collecting material removed from a surgical site, the attachment comprising: an attachment body; an attachment inlet couplable with an arthroscope instrument configured for removing material from the surgical site by suction; and an attachment outlet configured for fluid communication with a vacuum pump; wherein the attachment inlet and attachment outlet are arranged such that in use, application of a vacuum at the attachment outlet draws an airstream containing material from the surgical site into the attachment inlet, and liquid and solid material in the airstream drop into the attachment body.

In some embodiments, the attachment inlet and the attachment outlet are provided on an upper portion of the attachment.

In some embodiments, the attachment inlet comprises a channel extending inside the attachment body such that in use, the airstream from the surgical site is required to change direction before exiting the attachment through the attachment outlet.

In some embodiments, the attachment comprises an attachment lid which is removable from the attachment body. One or both of the attachment inlet and the attachment outlet may be provided through the lid.

In some embodiments, the attachment may comprise an attachment container which is removably couplable with the attachment body and into which the liquid and solid material is collected when in use.

An aspect of the present disclosure also provides the method according to the foregoing, or an apparatus according to the foregoing, used with or incorporating to the attachment.

It is to be understood each of the various aspects described herein may incorporate features, modifications and alternatives described in the context of one or more other aspects and embodiments/examples, such as but not limited to the apparatus, kit and methods for performing tissue repair and compositions produced using the apparatus, kit and/or methods. For efficiency, such features, modifications and alternatives have not been repetitiously disclosed for each and every aspect and embodiment/example although one of skill in the art will appreciate that such combinations of features, modifications and alternatives disclosed for some aspects and embodiments/examples apply similarly for other aspects and embodiments/examples and are within the scope of and form part of the subject matter of this disclosure.

Various aspects of the present invention and further embodiments of the aspects described in the preceding paragraphs will become apparent from the following description, given by way of examples and with reference to the accompanying drawings.

### BRIEF DESCRIPTION OF DRAWINGS

The present invention will now be described in greater detail with reference to the accompanying drawings. It is to be understood that the embodiments shown are examples only and are not to be taken as limiting the scope of the invention as defined in the provisional claims appended hereto.

FIG. 1 is a schematic illustration of an apparatus for preparation of a treatment composition comprising a filter barrel inserted inside a collection vessel.

FIGS. 2a to 2d are a top, side, bottom and sectional view (taken through the line A-A in FIG. 2b), showing a schematic illustration of the filter barrel of FIG. 1.

FIG. 3 is a schematic illustration of an apparatus for preparation of a treatment composition comprising a strainer attached to or formed integrally with an interior of a collection vessel.

FIGS. 4a to 4d are a side, perspective, top and sectional view (taken through the line A-A in FIG. 4c), showing a schematic illustration of an alternative filter barrel arrangement inside a collection vessel. FIG. 4e is an enlarged view of the portion marked B in FIG. 4d.

FIG. 5a is an isometric view of an apparatus for preparation of a treatment composition comprising a filter barrel comprising elements assembled inside a collection vessel. FIG. 5b is an exploded view of the apparatus of FIG. 5a. FIG. 5c is a side view of the filter barrel and a spacer forming part of the apparatus of FIGS. 5a and 5b. FIG. 5d is an end view of the filter barrel showing a strainer. FIG. 5e is a sectional view of the filter barrel and spacer of FIG. 5c, taken through line A-A.

FIG. 6 is an isometric view of a connector and collection container couplable with the apparatus of FIGS. 1 to 5d.

FIG. 7 is an isometric view of the apparatus of FIGS. 5a to 5e coupled with the collection container of FIG. 6 with a centrifuge cap applied over.

FIG. 8a is an isometric view of the assembly of FIG. 7 inserted within a centrifuge tube. FIG. 8b is a top view of FIG. 8a. FIG. 8c is a sectional view taken through the line A-A in FIG. 8b.

FIG. 9a is an isometric view of an attachment for receiving harvested tissue during an arthroscopic procedure. FIG. 9b is a top view of the attachment of FIG. 9a. FIG. 9c is a sectional view taken through the line B-B in FIG. 9b.

FIG. 10 is a schematic representation of a method 500 for preparing a treatment composition involving use of a collection vessel that is preloaded with a volume of functionalised polymer.

FIG. 11 is a schematic representation of a method 600 for preparing a treatment composition involving use of a delivery vessel that is preloaded with a volume of functionalised polymer.

FIGS. 12a and 12b are a schematic representation of a method 700 for preparing a treatment composition involving use of a product vessel that is preloaded with a volume of functionalised polymer.

FIGS. 13 to 16 are schematic illustration of kits for use in preparation of a treatment composition.

FIG. 17 illustrates a histological analysis of samples taken from cartilage defects 1 month after surgery, in accordance with Example 1, showing the effect of the cellular and biomaterial composition delivered at 50% in bioscaffolds made of GelMa 10%, LAP 0.1%, photocrosslinked at 20 mW/cm² for 1 min. a) Images show a comparison between native cartilage (“Cartilage”), cartilage with a 4 mm diameter lesion generated with a biopsy punch left untreated (“Empty”), or cartilage with a 4 mm diameter lesion generated with a biopsy punch and treated with the cell loaded polymer composition (“SVF”). From left to right: fibrillar collagen organisation detected with Second Harmonic Generation microscopy (SHG); the presence of Type II Collagen in the repaired area defect; haematoxylin eosin staining (H&E); and a macroscopic image of the treated areas. b) Quantification of the collagen fibers in the repaired area. c) ICRS macroscopic score for each treatment, performed by 4 independent unbiased assessors. d) Thickness of the repaired area. For each of b) to d), error bars indicate standard deviation and t-test statistical analysis indicates significance with a p value <0.005.

FIG. 18 illustrates a cell loaded polymer composition produced according to Example 2. Top, left to right: the cell loaded polymer composition was delivered in PDMS moulds and solidification was achieved via photocrosslinking with light using 405 nm light source at 20 mW/cm2 for 1 minute. The magnified area shows the immunostaining analysis performed on the whole bioscaffolds right after solidification (Day 0), using DAPI to stain the cell nuclei and anti-collagen type II. The histograms show the quantification of glycosaminoglycan and DNA performed on the bioscaffolds at day 0 and after 21 days of culture using papain extraction followed by DMMB quantification (GAG) and Pico Green quantification (DNA) (as outlined in Onofrillo, et al., 2021, Biomaterials, 264, 120383). Bottom, left to right: representative brightfield image of the bioscaffold after 21 days in culture. The dotted regions from 1-3 of the brightfield image indicate extracellular matrix accumulation areas that are positive for collagen type II staining (shown in panels 1 to 3).

FIG. 19 illustrates the cellular fraction (i.e., SVF) retrieved following the procedure of Example 3. a) Violin plot showing the distribution of the mass of cellular fraction product obtained using the arthroscope connected apparatus 100 from 16 fat pads of 16 different patients. b) Violin plot showing the quantification of the growth factor FGF-2 via ELISA test among 16 cellular fraction products obtained by 16 different patients. C) Metabolic activity of human adipose derived stem cells treated with cellular fraction product at different concentrations (0.1% to 10%) in comparison to a negative control with 0.9% NaCl (saline), measured by Cell Titer Blue Assay (Promega) following the manufacturer's instructions. For each of a) to c), error bars represent standard deviation and t-test was applied to evaluate significant difference between the groups with a p value <0.005.

### DETAILED DESCRIPTION

The present disclosure relates to use of novel apparatus, kits, systems and methods for preparation of a treatment composition comprising a cell loaded polymer composition that can be injected locally to deliver the cells to the site of injury and can subsequently provide an environment for cell ingrowth and tissue repair. Advantageously, the present disclosure enables both preparation and delivery of the treatment composition to the site of defect or injury to be performed in a single sterile environment in a single surgical procedure without the need for cell expansion. Resultant benefits, include simplification of the procedure as well as reduction in time, cost and risk of contamination.

The term “polymer” as used herein refers to homopolymers (formed by polymerisation of a single monomer species) and co-polymers (formed by polymerisation of a plurality of different monomer species), including linear polymers and cross-linked polymers. In some embodiments, the polymer can be a non-crosslinked or sparsely cross-linked polymer. In some embodiments, the polymer can be crosslinked.

The term, “functionalised polymer” refers to a polymer in which at least a portion of the individual monomer units are substituted with a specific functional group, for example, a functional group such as a methacryloyl group, that facilitates irreversible cross-linking of the polymer. For the functionalised polymers of the present invention, at least 1% or more, at least 2% or more, at least 5% or more, at least 10% or more, at least 15% or more, at least 20% or more, at least 30% or more, at least 50% or more, at least 75% or more, or at least 90% or more of the portion of the monomer units or reactive groups substituted with a specific functional group. The functionalised polymer may be capable of undergoing a phase change. In this context, ‘phase change’ will be understood to refer to a change in physical state, for example, a change to a solid (including gel) or a liquid phase. In some embodiments, the phase change may be reversible. A phase change from solid to liquid increases the flowability of the functionalised polymer to, for example, allow for delivery of the polymer and/or mixture with the cellular fraction (e.g. SVF) or extract obtained from the harvested tissue. A phase change from liquid to solid decreases the flowability of the functionalised polymer to, for example, to make it suitable for storage or transport or to increase the shelf life prior to mixture with the cellular fraction (e.g. SVF) or extract, or to allow for cell in growth and tissue repair or regeneration following administration of the cell loaded polymer composition to the tissue defect.

An example of a functionalised polymer suitable for use with some embodiments disclosed herein is described in International patent application PCT/AU2022/051439 the entire contents of which is hereby incorporated herein by this reference.

An “amine reactive group” or “hydroxyl reactive group” or “carboxyl reactive group” can be any functional group able to react with an amine group, or hydroxyl, or carboxyl group, respectively.

In some embodiments, the polymer is alginate. For example, alginate having a M/G ratio of about 1.30. In some or further embodiments, the alginate has a molecular weight of about 270 kDa. The alginate can be reversibly crosslinked when combined with an ionic crosslinking agent, preferably a divalent cation, more preferably Ca2+.

In some embodiments, the polymer is functionalised with a plurality of photocrosslinkable moieties (e.g., methacryloyl groups reacted with a hydroxyl group, a carboxyl group or an amine group of the polymer, for example, methacrylate groups reacted with a hydroxyl group of alginate) capable of crosslinking when combined with a photoinitiator and exposed to light.

In some embodiments, the functionalised polymer is methacrylated alginate with, for example, approximately 45-46% methacrylate functionalisation.

In some embodiments, the polymer is functionalised with a plurality of cell adhesion moieties, for example a peptide with cysteine and/or thiol (—SH) functionality. In some embodiments, the peptide comprises an integrin binding motif, for example, an Arg-Gly-Asp (RGD) (SEQ ID NO:1), RGDS (SEQ ID NO:2), GGGGRGDSP (SEQ ID NO:3), GRGDSP (SEQ ID NO:4), or GRGDS (SEQ ID NO:5), or an amino acid sequence with 1 or 2 amino acid insertions, deletions, substitutions (preferably conservative substitutions) or a combination thereof, typically outside the RGD motif. The peptide for cell adhesion may preferably comprise a cysteine residue and a RGD cell adhesion motif, for example, CRGDS (SEQ ID NO:6).

In some embodiments, RGD-based peptide sequences are introduced into methacrylated alginate via a thiol-Michael addition (e.g., methacrylate groups reacted with peptide to produce methacrylated alginate-RGD with for example, 5-6% RGD, as a wt % substitution of reactive groups).

In some embodiments, the functionalised polymer is about 270 to about 280 kDa alginate with M/G ratio of approx. 1.3-1.4, about 40-60% (preferably 40-50%, e.g., 45-46%, as a % of functionalised monomers) methacrylate functionalisation and 5-6% RGD (as a wt % substitution of reactive groups.

In other embodiments, the polymer is a bovine or porcine gelatine functionalised with methacryloyl groups, designated gelatin methacryloyl (GelMA). In some embodiments, the GelMA has between about 80 and about 90% methacryloyl functionalisation, for example, about 84% methacryloyl functionalisation.

In one embodiment, the functionalised polymer is Type A gelatin obtained from porcine skin, bloom 300, with from about 80% to about 90% methacryloyl functionalisation, for example, about 84% methacryloyl functionalisation.

As used herein, a “polymer composition” can comprise one or more polymers and/or functionalised polymers whether crosslinked or not, solubilised in solvent (e.g., aqueous medium such as water or saline solution) or solvated in solvent. The polymer composition can also comprise, for example, one or more divalent cations, more preferably Ca2+; one or more chelators such as EDTA; and/or one or more photoinitiators (e.g., c lithium phenyl-2,4,6 trimethylbenzoylphosphinate (LAP)). The polymer composition can be in a solid (including a gel state) or a liquid state.

In some embodiments, the polymer composition has a concentration of, for example, from about 4% to about 20% (w/v) GelMA, preferably about 12% to about 16% (w/v) GelMA, for example about 6%, 8%, 10%, or 20% (w/v) GelMA.

In some embodiments, the polymer composition has a concentration of, for example, from about 4% to about 20% (w/v), preferably about 8% to about 10% (w/v) methacrylated alginate-RGD in solvent.

In some embodiments, the polymer composition may also comprise the photoinitiator, lithium phenyl-2,4,6 trimethylbenzoylphosphinate (LAP) at a final concentration of, for example, 0.05% or 0.1% or 0.2% (w/v).

Cross-linking a polymer composition can change the state of the polymer composition from a “liquid” to a “solid” or “gel”. The cross-linking may or may not be reversible. Cross-linking can be used to modify the behaviour and/or mechanical properties of the resulting polymer networks, for example, modify, the viscosity, solubility, mechanical strength, stiffness, and/or rigidity.

In general, polymer networks can be obtained by either chemical or physical (e.g., ionic cross-linking). The cross-links are formed by covalent chemical bonds, or by physical interaction (e.g., ionic bonds), respectively.

In order for the polymer composition to have a sufficient cross-linking capacity, it is preferred that one or more of the monomers of the polymer(s) comprise cross-linkable groups. Preferably, such cross-linkable groups are selected from: hydroxyl groups, acid groups, aldehyde groups, carbonyl groups, amine groups, and oxirane groups. Preferably, these functional groups are derived from esters or amides of methacrylic acid.

Physically cross-linked networks possess temporary connections either through polymeric chain entanglement or physically induced gelation through, for example, ionic interaction, hydrogen bonding, thermo-induced gelation, complementary binding, inclusion complex formation, and hydrophobic interactions. Physical cross-linking can be reversed by for example a change in pH, temperature, ion concentration, or addition of a chelator such as EDTA).

Chemically crosslinked networks are typically not reversible. This can be achieved, for example, by thermal polymerisation, photopolymerisation, or enzymatic crosslinking. Generally, crosslinking through photopolymerisation involves polymers with vinylic such as methacryloyl groups, which allow for photopolymerisation. The vinylic double bond renders the compound highly sensitive to free radicals generated by an initiator under UV radiation and consequently to chain polymerisation.

Some covalent interactions are “reversible”, these are often called “dynamic covalent interactions” and include Schiff base reaction, click chemistry, and Michael addition. As such, “chemically cross-linked networks” as used herein can refer to “permanent” or “reversible” networks.

Change in state from solution (also referred to herein as “liquid”) to solid or gel, or vice versa, can be referred to as a “phase change” or “phase transition”. In this context, “phase change” or “phase transition” will be understood to refer to a change in physical state, for example, a change to a solid (including gel) or a liquid phase. And the change may or may not be reversible. Static covalent cross-links are not reversible.

In some embodiments, a phase transition in the cross-linked polymer chain conformation can be induced that leads to a change of the properties of the macroscopic network. For example, the phase transition may change the viscosity of the polymer composition and its flow properties to make it suitable for, for example, injectability, or to improve its draw rate. For example, a phase change from solid to liquid may increase the flowability of the functionalised polymer to, for example, allow for delivery of the polymer and/or mixture with the cellular fraction (e.g. SVF) or extract obtained from the harvested tissue. In other examples, a phase change from liquid to solid may change the viscosity of the polymer composition and its flow properties to make it suitable for storage or transport or to increase the shelf life.

Postdelivery to the defect site, the polymer composition can be cross-linked for in situ stabilisation of the polymer composition as a function of, for example, chemical agents, physiological stimuli (such as temperature, pH and ionic concentration) or light (which requires the introduction of a photoinitiator) to, for example, allow cell in growth and tissue repair or regeneration.

Polymer compositions and cross-linking strategies envisioned for biomedical applications should be biocompatible, and preferably, they (i) should not trigger an excessive inflammatory response, (ii) be biodegradable within a desired time frame (unless envisioned as permanent implant), (iii) be cleared from the body without the production of toxic byproducts, and (iv) provide the appropriate environment for cell proliferation and tissue growth.

Preferably, polymer compositions for cartilage regeneration (i) allow for ease of administration under physiological conditions, (ii) are injectable (with gelation following injection via either chemical or physical cross-linking), (iii) biocompatible and potential biodegradable, (iv) mimic cartilaginous extracellular matrix features and promote chondrogenic potential of cells, (v) fill defect sites inside the joint and integrate with the surrounding native cartilage tissue rather than shifting readily and (vi) have a sustained release profile if associated with local drug delivery.

The polymer composition may be a hydrogel.

“Hydrogels” are a class of polymer materials that possess high water content and elastic properties with cross-linked (e.g., through covalent bonds or held together via physical intramolecular and intermolecular attractions), multiporous networks. Hydrogels can be classified into different categories based on various parameters such as preparation method, ionic charge, and mechanical and structural characteristics.

Hydrogels can be broadly classified based on the source material (natural or synthetic) and biodegradability (biodegradable or non-biodegradable). Natural hydrophilic macromolecules used for hydrogel scaffold fabrication are often biodegradable and mainly consist of proteins and polysaccharides. Natural polymers commonly used for the formation of hydrogels include gelatin, collagen, hyaluronic acid, alginate, fibrin, and chitosan, while synthetic polymers include poly(ethylene glycol) (PEG), poly(vinyl alcohol) (PVA), polydioxanone, poly(lactic acid) and poly(N-isopropylacrylamide). These polymers can be functionalised with one or more functional groups to make physically or chemically cross-linked hydrogels.

Any technique which can be used to create a cross-linked polymer can be used to produce a hydrogel. Common noncovalent chemical interactions used for the design of hydrogels include electrostatic, metal coordination, hydrophobic, and hydrogen bonding. Covalent crosslinking can be done through numerous approaches including through chemical reactions of complementary groups.

For biomedical applications, chemical reactions employed in cross-linking hydrogels are preferably achievable in aqueous solutions without generating toxic byproducts. The reaction is preferably efficient with few reactants and active functional groups remaining. For that purpose, click chemistry, Michael additions, thiol-ene/yne coupling, Diels-Alder reaction, disulfide formation, Schiff-base formation, and epoxide reactions are examples of suitable reactions that can be used.

A number of hydrogel-based scaffolds have been developed that can be used in cartilaginous tissue engineering, and sufficient mechanical properties for repairing cartilage defects to restore normal joint function. To further enhance the mechanical properties of scaffolds, traditional single-network hydrogels can be supplemented with either additional networks or mixtures of polymers (including functionalised polymers). Nanocomposites can also be utilised to vary the mechanical properties of scaffolds to produce hydrogels which can improve the integration with surrounding cartilage while promoting chondrogenesis of stem cells encapsulated in hydrogels in vivo.

In the present disclosure, a polymer composition, for example, an hydrogel, can be used as carrier for live cells (e.g., live stem or progenitor cells) to deliver the cells to the defect site and stimulate tissue repair or regeneration. Examples of cells that can be incorporated into the polymer composition include one or more of chondrocytes, progenitor cells and stem cells (e.g., endothelial progenitor cells (EPCs), mesenchymal stromal cells (MSCs), induced pluripotent stem cells (iPSCs) and predifferentiated MSCs).

In preferred embodiments, a cellular fraction or extract thereof, for example, obtained from a harvested tissue is incorporated into the polymer composition, for example, an hydrogel. In some embodiments, the cellular fraction or extract is obtained from adipose tissue. The cellular fraction may be a “stromal vascular fraction”. The stromal vascular fraction (SVF) may comprise one or more of the following cell types; adipocytes, fibroblasts, smooth muscle cells, endothelial cells, blood cells, EPCs, preadipocytes, vascular progenitors, hematopoietic progenitors, MSC, hematopoetic stem cells (HSC), pericytes, supra-adventicial cells. The cellular fraction or extract thereof may be diluted with, for example, a buffer solution (e.g., saline) prior to being mixed with the polymer composition to form a “cell loaded polymer composition”. The cell loaded polymer composition may also be diluted with, for example, a buffer solution (e.g., saline) prior to delivery to the defect site. In some embodiments, the cellular fraction (e.g., SVF) is diluted to about 5% to about 20% in a buffer solution prior to being mixed with the polymer composition, for example, at a 1:1 ratio. In one embodiment, the polymer composition is gelatin methacryloyl (GelMA) and on mixing with the cellular fraction (e.g., SVF), the GelMA is diluted to about 2% to about 10% (v/v), preferably about 6% to about 8% (v/v). In another embodiment, the polymer composition is methacrylated alginate-RGD and on mixing with the cellular fraction (e.g., SVF) is diluted to about 2% to about 6% (v/v), preferably about 4% to about 5% (v/v).

Advantageously, the present disclosure allows for control of the ratio of cellular fraction or extract thereof to polymer composition.

In some embodiments, the treatment compositions of the disclosure comprise one or more bioactive molecules, for example, one or more chemical mediators such as growth factors, interleukins.

In some embodiments, the polymer composition is in a solid or gel state prior to administration and a phase transition in the cross-linked polymer chain conformation is induced that leads to a change of state to a “liquid” form with lower viscosity allowing it to be mixed with the cellular fraction or extract and/or collected (e.g., drawn into a delivery vessel), and/or delivered to the defect site (e.g., from the delivery vessel by injection). However, that need not be the case, and the polymer composition may be supplied in a liquid state allowing it to be mixed with the cellular fraction or extract and/or collected without requiring a prior phase change.

The cell loaded polymer composition can, for example, be delivered to the defect site in an open surgery or non-invasive or minimally invasive manner by, for example, direct injection or arthroscopy. Preferably, the cell loaded polymer composition is of suitable viscosity to allow for homogenous delivery and distribution of the cells inside the tissue defect (e.g., cartilage defect).

Preferably on administration, the cell loaded polymer compositions can fill the defect area, preferably with a smooth interface (e.g., that is similar to the native cartilage), preferably without integrating into the surrounding healthy tissue. Preferably following in situ stabilisation, the cell loaded polymer compositions allow for cell in growth and tissue repair or regeneration of the defect.

Histological analysis of in vitro or ex vivo samples can be used to quantify the degree of tissue repair or regeneration. For example, haematoxylin and eosin (H&E) staining of nuclei and extracellular matrices, respectively, can report on aspects of the tissue cellular structure (for example, defect area thickness). A macroscopic score can also be calculated using the histological analysis, according to the ICRS Clinical Cartilage Injury Evaluation system-2000, cartilage injury evaluation standard (accessible at https://cartilage.org/content/uploads/2014/10/ICRS_evaluation.pdf). In some embodiments, a defect area treated with a cell loaded polymer composition of the disclosure will have a similar macroscopic score when compared with healthy tissue 1 month after treatment. In further or alternative embodiments, a defect area treated with a cell loaded polymer composition of the disclosure will have a greater macroscopic score than a defect area left untreated, 1 month after treatment.

Cellular imaging may also be used to assess structural characteristics of a defect area. For example, second harmonic generation (SHG) microscopy can be used to visualise the fibrillar type II collagen structures present in cartilage tissue. This may allow for quantification of the relative amounts of collagen in a region of interest (ROI); collagen fibre density; collagen reticulation index (a measure of how many collagen fibre branches there are within the entire length of the collagen network); and the number of collagen fibres per square millimetre. Macroscopic imaging of the entire defect area can also report on the overall tissue structure. In some embodiments, a defect area treated with a cell loaded polymer composition of the disclosure will have a similar relative amount of collagen in a ROI, collagen fibre density, collagen reticulation index, and number of collagen fibres per square millimetre, compared with healthy tissue 1 month after treatment. In further or alternative embodiments, a defect area treated with a cell loaded polymer composition of the disclosure will have a greater relative amount of collagen in a ROI, collagen fibre density, collagen reticulation index, and number of collagen fibres per square millimetre, when compared with a defect area left untreated, 1 month after treatment.

Treatment efficacy may also be assessed by quantifying the cellular processes that regulate cartilage degeneration, regeneration, and/or homeostasis after treatment. For example, fibroblast growth factor 2 (FGF-2) promotes regeneration of cartilage by attracting mesenchymal stem cells to the site of cartilage injury. Levels of FGF-2 may indicate the potential promotion of cartilage repair as a result of treatment, as FGF-2 that stimulates native cartilage repair; FGF-2 levels may be measured by any suitable technique known in the art (for example, enzyme-linked immunosorbent assay (ELISA)). In some embodiments, increased FGF-2 levels in cartilage defect areas treated with a cell loaded polymer composition of the disclosure, compared to untreated tissue, may indicate an increased repair potential. Another measure of treatment efficacy is the metabolic activity of cells at the defect area, after treatment with the cell loaded polymer composition, which may be measured by any suitable technique known in the art (for example, Cell Titer Blue Assay, Promega). In some embodiments, human adipose derived stem cells may have increased metabolic activity after treatment with a cell loaded polymer composition of the disclosure, compared to a saline treatment.

Where the terms “comprise”, “comprises”, “comprised” or “comprising” are used in this specification (including the provisional claims) they are to be interpreted as specifying the presence of the stated features, integers, steps or components, but not precluding the presence of one or more other features, integers, steps or components or group thereof.

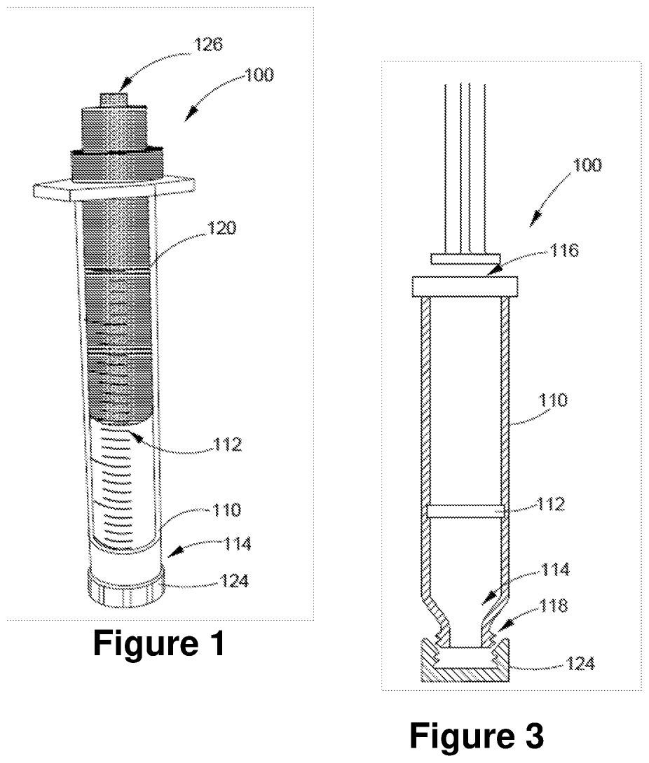

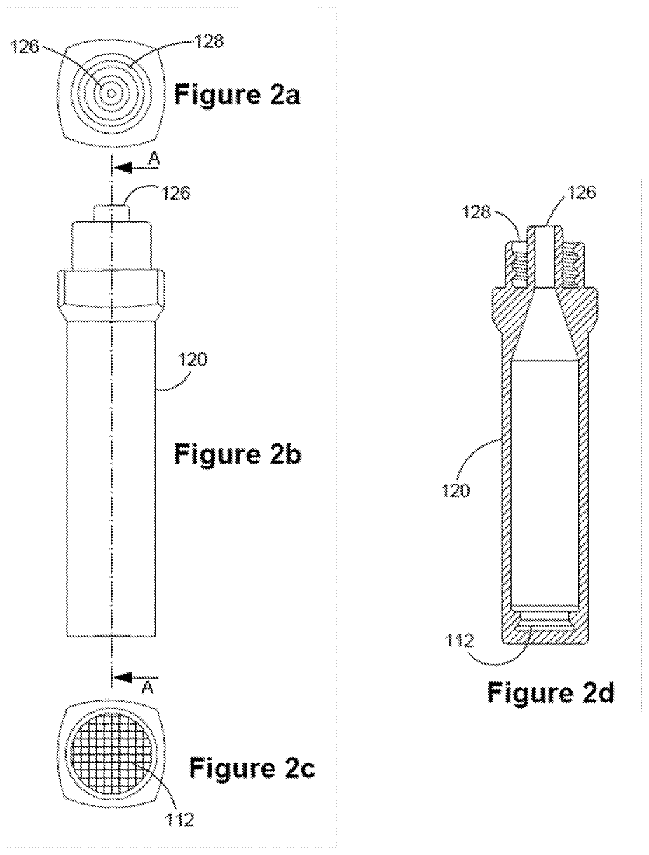

FIGS. 1 and 2 are schematic illustrations of an apparatus 100 for preparation of a composition for repair or regeneration of tissue, or treatment of a tissue defect according to embodiments of the disclosure. The apparatus 100 comprises a collection vessel 110 configured to receive harvested tissue and at least one filter element located inside the collection vessel to assist with separation of a cellular fraction, for example, a Stromal Vascular Fraction (SVF) fraction, from the harvested tissue within the collection vessel. The filter element comprises a filter member or strainer 112 located inside the collection vessel 110 when in use so as to aid in separation of the cellular fraction (e.g., SVF) from the harvested tissue.

In the embodiment shown in FIG. 1, the filter element comprises a hollow filter barrel 120 with a strainer 112 at one end. The filter barrel 120 is insertable into the collection vessel 110 such that the strainer 112 sits inside the collection vessel when assembled. Harvested tissue can be loaded into the filter barrel through opening 126. In the embodiment shown, this is achieved by coupling a syringe or other device (not shown) containing the harvested tissue with the filter barrel opening 126 via a luer coupling 128 and advancing a plunger into the syringe to load the harvested tissue into the filter barrel. FIGS. 2a to 2d are a top, side, bottom and sectional view (taken through the line A-A of FIG. 2b) of the filter barrel of FIG. 1. The top and sectional views in FIGS. 2a and 2d show the threaded luer coupling 128 for attachment of a syringe containing the harvested tissue. The bottom and sectional views in FIGS. 2c and 2d show the strainer 112. Collection vessel 110 may comprise a bottom opening 118 having a narrowing or neck that is couplable with e.g. a delivery device 140 or a valve connector 150 via a luer coupling or the like. A removable stand 124 may be provided over the bottom opening 118 to retain the collection vessel 110 in an upright condition.

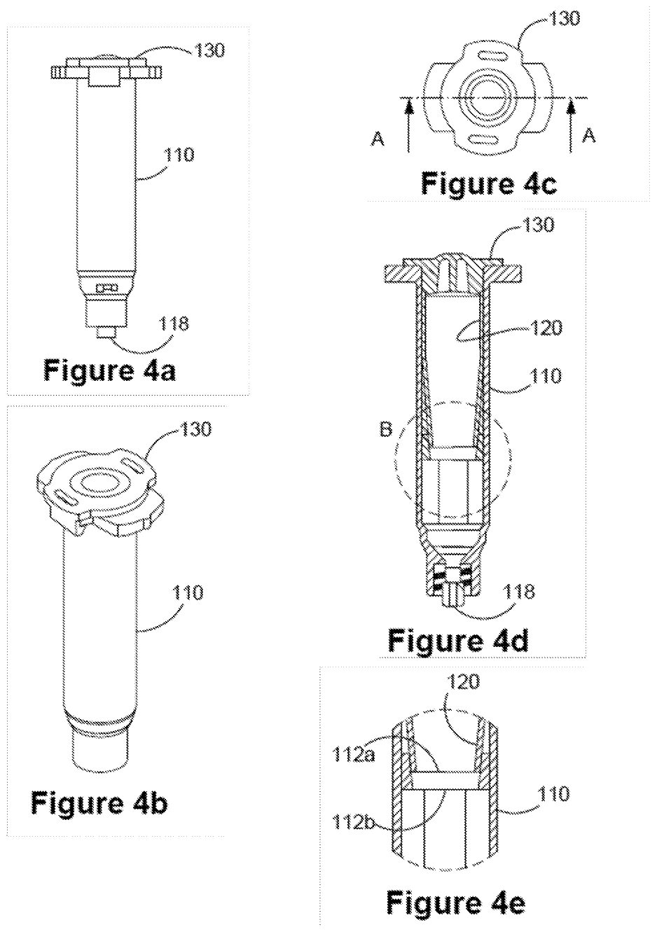

In the embodiment shown in FIG. 3, the filter element comprises a strainer 112 attached to, or formed integrally with, an interior wall of the collection vessel 110. Harvested tissue may be loaded directly into the top opening 116 of the collection vessel, atop the strainer 112. An advantage of providing the filter element or strainer 112 attached to or formed integrally with an interior wall of the collection vessel includes reduction of parts and materials, and the ability to sterilise the apparatus as a single piece. In some embodiments, the apparatus comprising the collection vessel 110 may be manufactured with the interior filter element/strainer integral to the vessel using a sterile single process such as injection moulding which may reduce cost associated with materials. In addition to the cost benefit associated with requiring fewer parts, eliminating the process of transferring harvested tissue from a filter barrel to the collection vessel interior may reduce loss of harvested tissue which may be important when dealing with small volumes.

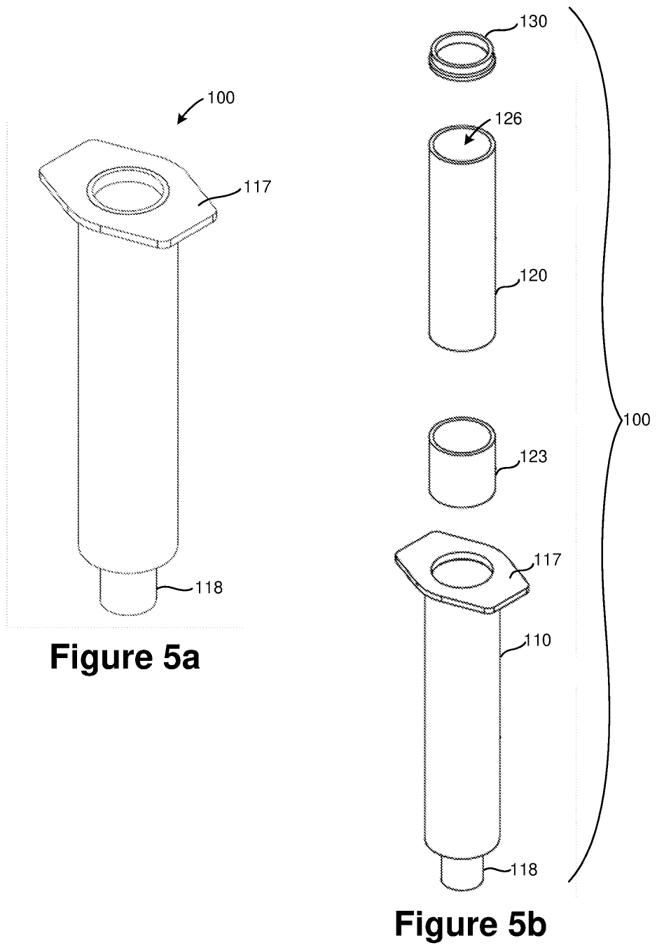

Another example of an apparatus 100 comprising a collection vessel 110 and a filter barrel 120 is provided in FIGS. 4a to 4e. In this embodiment, filter barrel 120 is open at a top end and a removable closure 130 is provided to close the filter barrel after receiving the harvested tissue to avoid loss of materials, for example as the apparatus containing the tissue is transferred into the centrifuge. Additionally, two filter elements 112a, 112b are provided in filter barrel 120 as shown in the enlarged sectional view of FIG. 4e. It is to be noted that the two filter elements need not be arranged proximal one another as shown. In some embodiments they may be spaced further apart such that one filter element 112b is arranged e.g. at or near the end of the filter barrel 120, and the other filter element 112a positioned somewhere between e.g. the mid point of the filter barrel and the first filter element 112b.

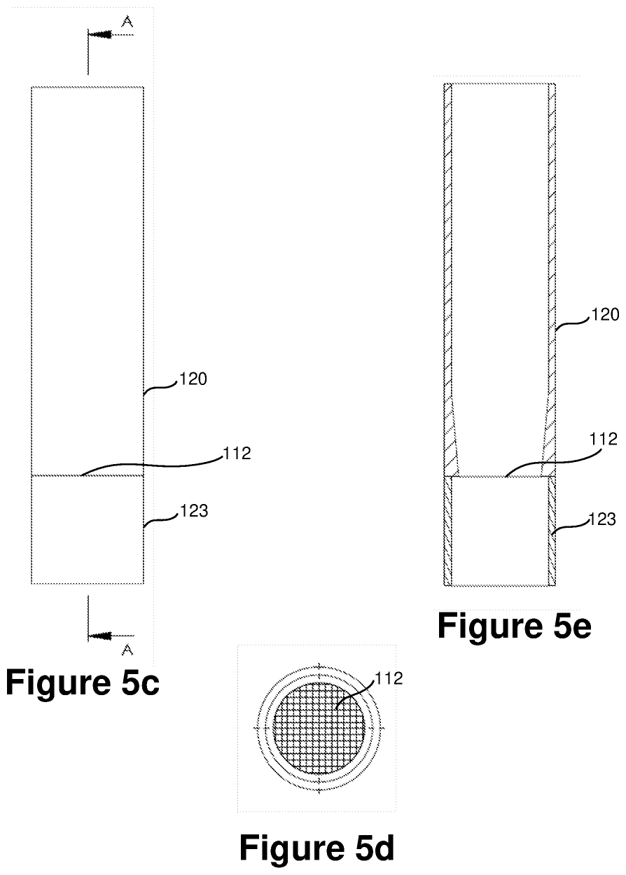

Another example of an apparatus 100 comprising a collection vessel 110 and a filter barrel 120 is provided in FIGS. 5a to 5e. The filter barrel 120 comprises a strainer 112 which may be located inside or at/toward one end of the filter barrel which is insertable into the collection vessel 110 such that the strainer 112 sits inside the collection vessel when assembled. In some embodiments, the apparatus 100 may be provided pre-assembled, which may streamline use of the apparatus in preparation of a treatment composition. FIG. 5a shows the assembled open apparatus 100. In the embodiment shown, strainer 112 is arranged at one end of the filter barrel 120. A spacer 123 may be provided to create a space for the cellular fraction (e.g. SVF) to move through the apparatus during centrifuging and reduce the likelihood of the cellular fraction becoming caught inside the collection vessel 110. It is to be understood however that this function need not be provided by a separate spacer element and that spacing may be achieved by e.g. a support member formed on the inside of the collection vessel 110 onto which the filter barrel 120 may rest or become fixed during assembly. In another example, the internal surface and/or diameter of the collection vessel 110 (and/or the external surface of the filter barrel 120) may be modified to retain the filter barrel 120 within the collection vessel 110 e.g. by friction fit, such that the filter barrel end and strainer 112 is spaced from the bottom opening 118. The strainer 112 is best shown in FIGS. 5d-e. In some embodiments, strainer 112 may be set back inside the filter barrel body 120, obviating the need for a spacer 123. In some embodiments, more than one strainer may be provided in the filter barrel 120, with or without a spacer 123.

To assemble the apparatus 100 as shown in FIGS. 5a-e, the spacer 123 is inserted into the collection vessel 110 followed by the filter barrel 120. In some examples, the filter barrel 120 may be retained within the collection vessel 110 by a friction fit to limit the risk of disassembly and/or e.g. loss of harvested tissue during transfer into a centrifuge and/or use of the apparatus to prepare a treatment composition. Alternatively or additionally, the apparatus may comprise a removable closure 130 which can be applied to the open end 126 of the filter barrel to prevent loss of contents. In some embodiments the closure 130 (FIG. 5b) may also retain the filter barrel 120 within the collection vessel 110 although a separate removable closure may be provided for this purpose. In some embodiments the collection vessel 110 may comprise a grasping portion such as flange 117. Collection vessel 110 may comprise a bottom opening 118 that is couplable with other components that may be useful for preparation of the treatment composition. The opening 118 may comprise a narrowing or neck as shown to couple with other components e.g. by luer lock, threaded or friction fit coupling or the like. Such components may include but are not limited to e.g. a closure to stop leakage of contents, a collection container, a delivery vessel or a mixing vessel for preparation of the treatment composition.

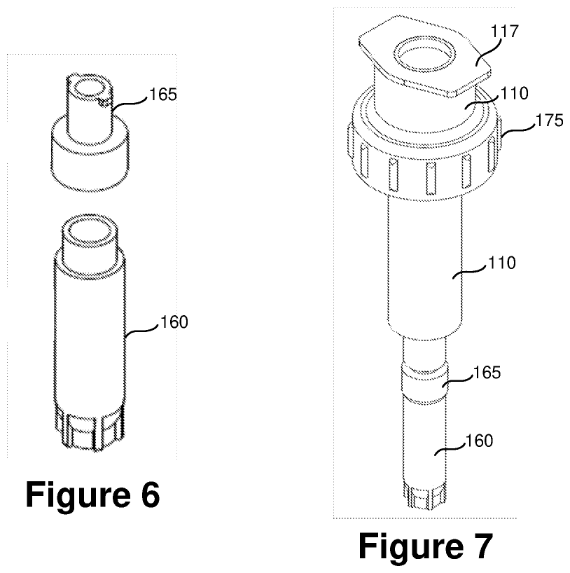

FIG. 6 is an example of a collection container 160 which may be coupled directly or indirectly, e.g. via a coupler 165 with the bottom opening 118 of the collection vessel 110. A collection container 160 may be utilised to collect the cellular fraction (e.g. SVF) after centrifuging of the harvested tissue in the apparatus 100. In some embodiments it may be beneficial to collect the cellular fraction in a collection container 160 rather than in the base of the collection vessel 110 so that a portion of the contents may be preserved for later use, either in the same procedure or in a subsequent procedure. Thus in some embodiments collection container 160 may be couplable with a closure (not shown) for retaining the sterile contents comprising the collected cellular fraction (e.g. SVF).

The apparatus 100 may comprise a coupling (e.g. luer coupling 128 as in FIGS. 1, 2a-2d) for releasable attachment of a syringe or other device containing the harvested tissue from which the cellular fraction (e.g., SVF), is to be separated. Alternatively, the harvested tissue can be provided into the hollow body (e.g. via opening 116 as in FIG. 3) of the collection vessel 110, above the strainer 112, without such a coupling.

While a single strainer 112 is shown in the embodiments of FIGS. 1, 2 and 5a-e, it is to be noted that more than one strainer or filter element may be provided to aid in separation of the cellular fraction (e.g., SVF) from the harvested tissue. Ideally each strainer or filter element comprises a plurality of openings each having a cross-sectional dimension of about 30 to 60 um, preferably about 40 μm. Each strainer or filter may comprise a nylon or other material. In some embodiments, each strainer or filter element comprises a membrane having a molecular weight cut-off ranging from about 50 to about 500 kDA for separation of the cellular fraction (e.g., SVF) from the harvested tissue.

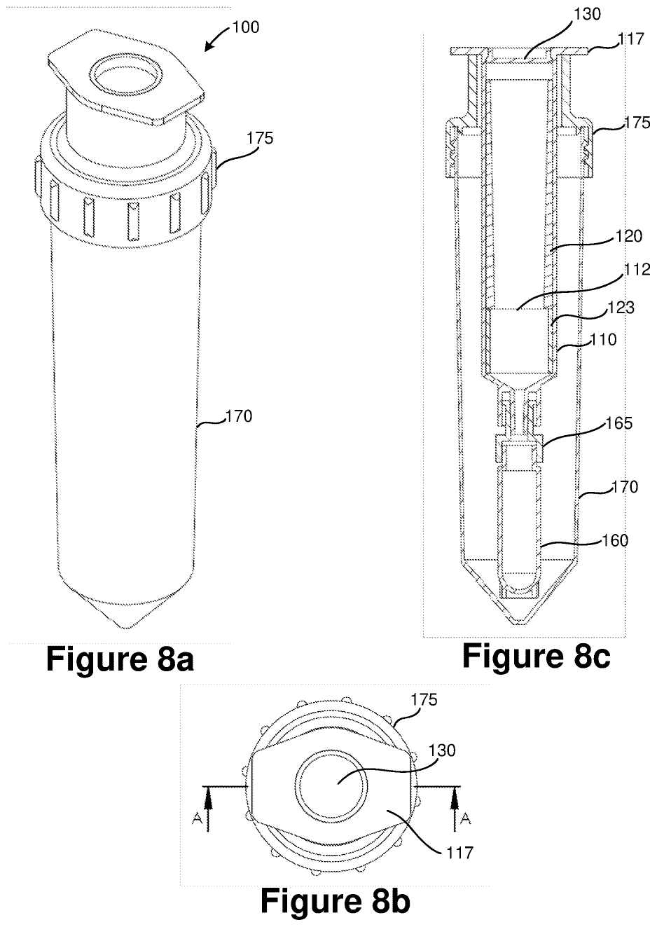

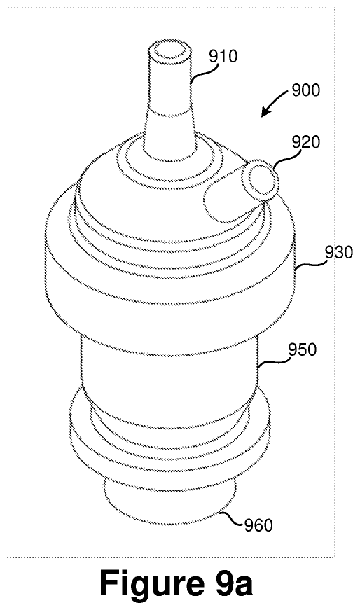

The apparatus 100 containing the harvested tissue is configured to be receivable in a centrifuge where the process of centrifuging can be undertaken to separate the cellular fraction (e.g., SVF) through the filter element 112 and into the bottom part 114 of collection vessel 110, or into a collection container 160 coupled with the collection vessel 110. FIG. 7 is an isometric view of the apparatus of FIGS. 5a to 5e coupled with the collection container of FIG. 6 with an open centrifuge cap 175 applied over. Cap 175 enables the centrifuge tube 170 to be used as a support vessel for the apparatus 100. This enables a standard centrifuge tube holder to be used to hold the apparatus 100. FIG. 8a is an isometric view of the assembly of FIG. 7 inserted within a centrifuge tube 170, such as a falcon tube. FIG. 8b is a top view of FIG. 8a. FIG. 8c is a sectional view taken through the line A-A in FIG. 8b. In some embodiments, the centrifuge tube 170 and centrifuge cap 175 may be threadedly engaged to secure the apparatus 100 containing the harvested tissue, for insertion into the centrifuge tube holder. Utilisation of the centrifuge tube 170 and cap 175 improves accommodation of the apparatus 100 within the centrifuge tube holder, while continuing to maintain sterility of the contents. It is to be understood that a centrifuge tube 170 may be utilised to receive the apparatus 100 within the centrifuge tube holder, with or without a collection container 160.

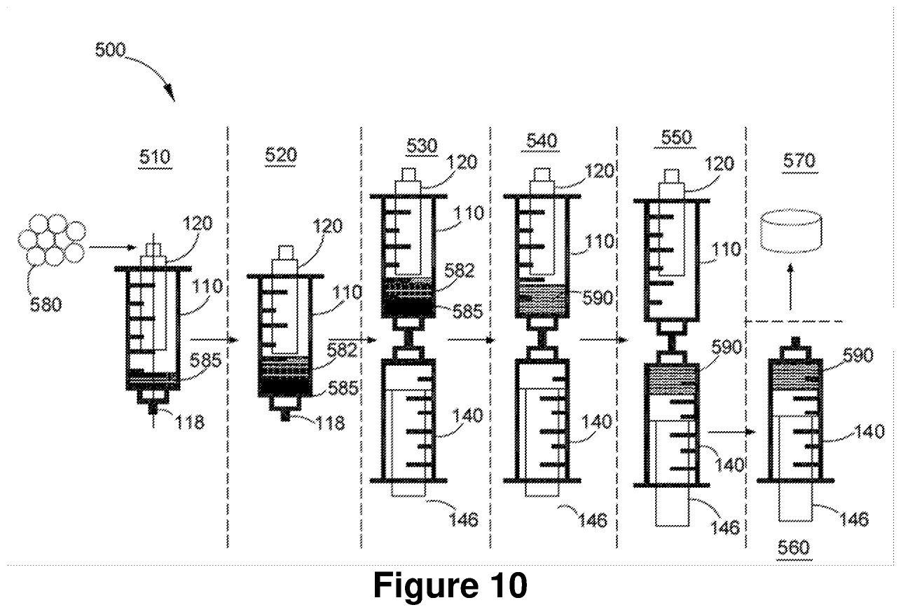

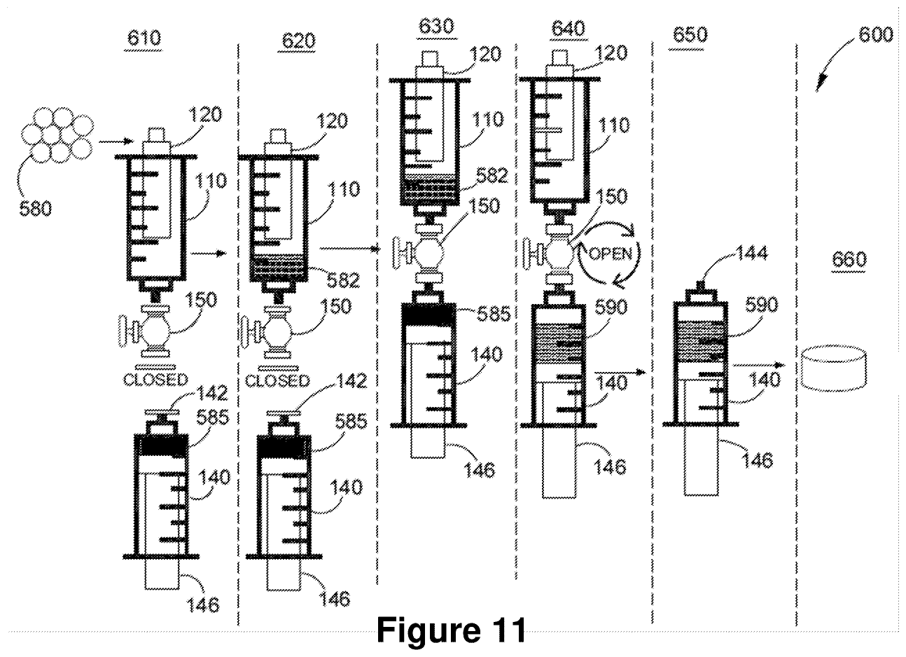

In some embodiments the apparatus may comprise or be coupled with a delivery vessel 140 (FIGS. 10 and 11). The delivery vessel 140 may be couplable with the collection vessel 110 using e.g. luer couplings. In some embodiments, the coupling between the collection vessel 110 and the delivery vessel 140 may comprise a valve as will be described with reference to FIG. 11. In some embodiments, the apparatus may comprise or be coupled with a delivery vessel 140 which is utilised to transport the cell loaded polymer composition to the site for repair.

In some embodiments, apparatus 100 is configured to receive a volume of a polymer composition for mixing with the cellular fraction (e.g., SVF) obtained from the harvested tissue. The polymer composition may be received or provided in either one of the collection vessel 110 and the delivery vessel 140 as described with reference to various embodiments herein. Alternatively, it is contemplated that part of the volume of the polymer composition may be received or provided in both the collection vessel 110 and the delivery vessel 140. In some examples, the polymer composition may be provided in a pre-loaded polymer container which is couplable with a vessel containing the cellular fraction (e.g. SVF). The cellular fraction (e.g. SVF) or extract may be mixed with a buffer e.g. saline solution to achieve a desired cell concentration.

A delivery vessel 140 may be supplied separately from or in a kit with the apparatus which comprises the collection vessel and filter element. In some embodiments, the collection vessel may be provided containing the polymer composition, and/or the delivery vessel may be provided containing the polymer composition. In some embodiments, a polymer container may be provided containing the polymer composition which can be combined with the cellular fraction (e.g. SVF) to provide the prepared cell loaded polymer composition, as will be discussed below.

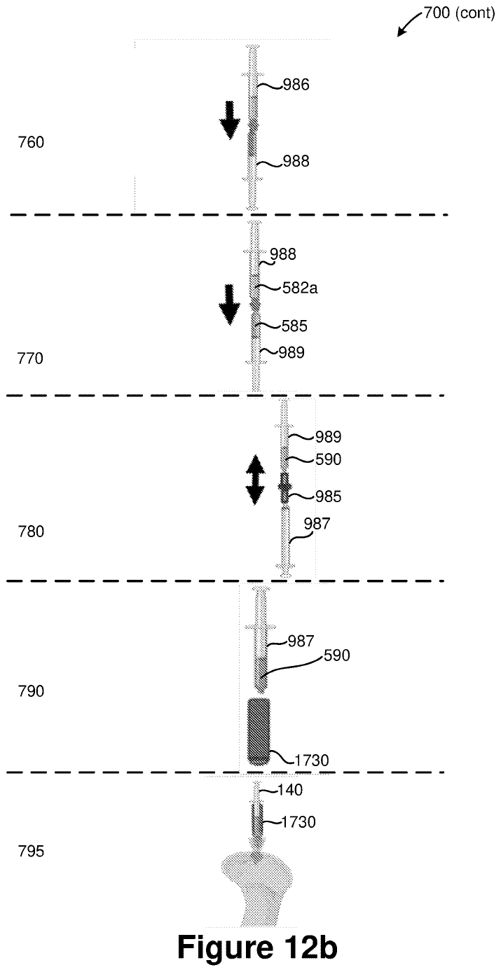

The delivery vessel 140 can receive the cell loaded polymer composition prior to delivery to the defect site for regeneration or repair of the tissue using a delivery device such as a biopen or the like (FIGS. 13 to 16). In other embodiments, the cell loaded polymer composition may be received directly into a delivery device from the collection vessel 110. In yet other embodiments still, the cell loaded polymer composition may be prepared inside the collection vessel 110 and delivered directly to the defect site as described below. An example of a biopen suitable for use as a delivery device is described in WO2018/053565A1, the disclosure of which is incorporated herein by this reference. However, it is to be understood that this is just one example of a delivery device, and that other devices may be suitable for delivery of the cell loaded polymer composition to the defect site.

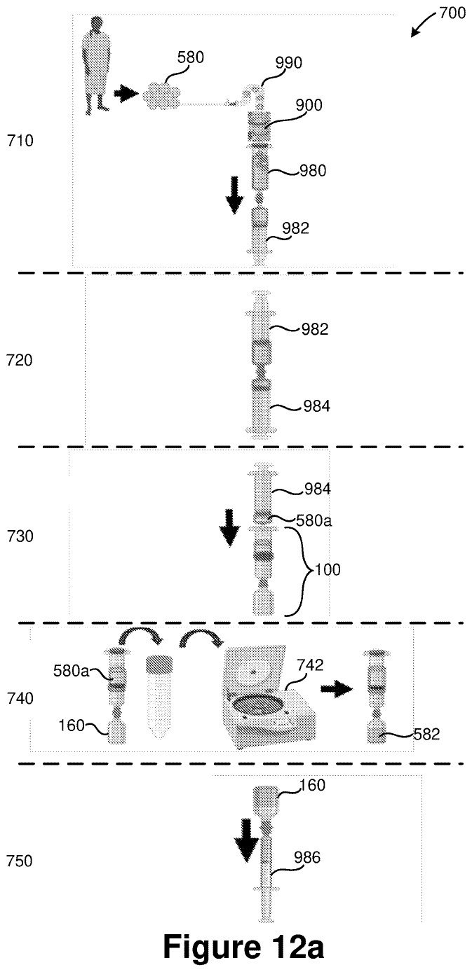

In some embodiments, the collection vessel 110 and/or the delivery vessel 140 may be pre-loaded with a volume of the polymer composition as will be described with reference to FIGS. 10 and 11. In some embodiments, a polymer container may be pre-loaded with a volume of the polymer composition as will be described with reference to FIGS. 12a and 12b. In some examples, the polymer container comprises a preloaded polymer syringe which may contain a known quantity of the polymer composition.

Use of the novel apparatus in the preparation of a treatment composition for repair or regeneration of tissue, or treatment of a tissue defect will now be described with reference to FIGS. 10 to 12b.

FIG. 10 is a schematic representation of a method 500 for preparing a treatment composition comprising a cell loaded polymer composition 590 according to an embodiment of the disclosure. In the embodiment shown, apparatus 100 of FIGS. 1 to 2d is employed however it is to be understood that the apparatus of FIG. 3 or 5a-e could be employed as alternatives. In the embodiment shown, the apparatus is provided with the collection vessel 110 preloaded with a volume of polymer composition 585 in the bottom part 114 (FIG. 1) of the collection vessel 110. Under typical conditions the polymer composition 585 is solid at about 20 deg C. to about 25 deg C.

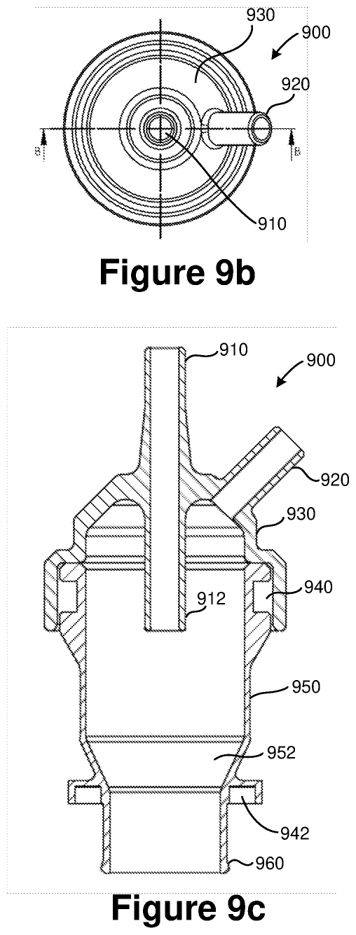

In a step 510, harvested tissue 580 is provided into the filter barrel 120. The tissue may be harvested arthroscopically or upon open surgery from e.g. the infrapatellar fat pad (IFP). IFP has been proven to be a good source for adipose derived stem cells e.g. mesenchymal stem cells for cartilage regeneration application. However, it is to be understood that harvested tissue may be from other regions. When tissue is harvested arthroscopically, the apparatus may be coupled with an attachment that provides for delivery of the harvested tissue from the arthroscope to the collection vessel 110 via a connected tubing system. An example of an attachment is provided in FIGS. 9a to 9c.

FIGS. 9a to 9c show an attachment 900 for collection of harvested tissue from an arthroscope. Traditional arthroscope traps capture all material removed during the arthroscopic process which is typically collected for disposal. There is no easy way to remove the material once it has been captured in the trap and maintain sterility. Attachment 900 allows the material removed during the arthroscope procedure to be captured into a collection device instead of being captured in the trap. This allows the captured biological material comprising the harvested tissue to be used in the preparation of the treatment composition according to embodiments of the present disclosure.

Attachment 900 provides an attachment inlet 910 and attachment outlet 920 which enables the attachment to be connected in the arthroscope suction path, upstream of the vacuum pump which applies suction to remove material from the surgical site. A suction tube (e.g. of approximately 10 cm length) from the arthroscope instrument is couped at attachment inlet 910, and a further tube (e.g. of approximately 100 cm length) is coupled between the attachment outlet 920 and a vacuum pump providing suction. The attachment 900 is designed to utilise the suction flow to draw material from the arthroscope instrument into the attachment body 950. During use of the attachment, the solid and liquid material in the suction path falls out of the airstream into the arthroscope attachment body 950. Owing to the design of the attachment 900, air entering the attachment must change direction before exiting the attachment outlet 920. Solid and liquid material in the flow path possess inertia which prevents acceleration by the airstream towards the attachment outlet 920 resulting in separation of the solid and liquid material which drops out of the suspension into the base area 952 of the attachment body 950 or a collection container which may be coupled thereto. In some examples, an inlet extension 912 protrudes into the attachment body 950. The inlet extension 912 may reduce risk of the attachment outlet 920 becoming occluded and/or may assist to direct solid and liquid material in the airstream into the attachment body base 952. Inlet extension 912 may also mitigate material being removed through the attachment outlet 920. A larger collection outlet 960 may be provided at the base of the attachment 900 for coupling with a collection device. To ensure sealing, a slot 942 may be provided for receiving a sealing member such as an O-ring, to ensure a sealing closure when a collection device is coupled with the collection outlet 960 of the attachment body 950. In some examples, the collection device may comprise the apparatus 100.

In some examples, the attachment inlet 910 and attachment outlet 920 are provided on an attachment lid 930 which may be coupled with the attachment body 950 e.g. by threaded engagement, friction fit or the like. To ensure good sealing for maintenance of suction pressure, slot 940 may be provided for a sealing member such as an O-ring, between the attachment body 950 and attachment lid 930. It is to be understood, however, that the attachment body 950 and attachment lid 930 may be formed as a unitary piece when a collection outlet 960 is provided for coupling with a separable collection container. The attachment 900 be comprised of any suitable material such as medical grade polypropylene or polycarbonate. In use, it may be preferred to utilise the attachment 900 in a substantially vertical orientation, or in an orientation not greater than about 45 degrees to vertical, for collection of the material comprising the harvested tissue. Unlike commercially available arthroscope traps (such as the Polyp Trap (Example 1, Example 2, Example 3) attachment 900 provides for efficient collection of the harvested tissue from which a precise volume can be transferred for preparation of the treatment composition. Commercially available traps do not provide a mechanism for direct connection to a collection device such as apparatus 100. With conventional traps, the entire trap needs to be opened to remove the collected material, which can be cumbersome and increase the risk of contamination and does not allow for precise volume retrieval.

In some embodiments, the method 500 may comprise processing the harvested tissue 580 to homogenise the sample prior to separation of the cellular fraction (e.g., SVF). Processing may involve e.g. transferring the harvested tissue between 2 syringes connected via a luer lock system, and/or via the action of an arthroscope. In some embodiments, one of the syringes contains the harvested tissue and the other syringe may contain a saline solution to assist with processing. It is to be understood however that the process of harvesting the tissue may achieve some degree of tissue processing/homogenisation such that an additional processing step(s) may not be required. An example of processing harvested tissue is described in relation to method 700 as shown in FIGS. 12a and 12b.

In a step 520, the cellular fraction (e.g., SVF) 582 is separated from the harvested tissue 580. This may be achieved by placing the apparatus 100 comprising the collection vessel 110 and the filter barrel 120 containing the harvested tissue 580 within a centrifuge (not shown) where centrifugation occurs to separate a cellular fraction (e.g., SVF) 582. The apparatus containing the harvested tissue may be centrifuged for about 3 to about 8 minutes at about 1800 to 2700 g, such as for about 5 minutes at about 2000 g using a standard laboratory centrifuge. In some embodiments, the collection vessel 110 may be configured to be received in a tube holder within the centrifuge. Alternatively, the collection vessel 110 may be configured to be received in a container such as a 50 mL centrifuge tube that is configured to be received in a tube holder within the centrifuge. At completion of separation step 520, the cellular fraction (e.g., SVF) 582 is separated out from the harvested tissue 580 and sits atop the polymer composition 585 in the bottom part 114 of the collection vessel 110. The polymer composition 585 in the collection vessel 110 acts like a plug, owing to its solid (e.g., gel) state, preventing loss of the cellular fraction (e.g., SVF) 582 from the collection vessel opening 118.

In a step 530, the collection vessel 110 is coupled e.g. via a luer coupling with a delivery vessel 140 prior to inducing a phase change in a step 540 to change the state of the polymer composition 585 from solid to liquid form of lower viscosity allowing it to be mixed with the cellular fraction. The phase change mechanism is selected based on the polymer composition. Once liquified, the polymer composition 585 is able to be mixed with the cellular fraction (e.g., SVF) 582. In one example provided below the pre-loaded polymer composition 585 comprises gelatin methacryloyl (GelMA), and the phase change mechanism comprises heating the apparatus to a target temperature (typically 37 deg C.). This may be achieved by placing the collection vessel 110 coupled with the delivery vessel 140 in an incubator, such as a standard laboratory incubator, to warm the contents. In another example provided below, the pre-loaded polymer composition comprises methacrylated alginate-RGD, and the phase change mechanism comprises addition of a solid to liquid phase change agent to the polymer composition, for example, a chemical agent (e.g., a chelator such as EDTA). The solid to liquid phase change agent may be introduced to the polymer composition via the delivery vessel 140 or via the top opening 116 in the collection vessel 110.

In a step 550, the cellular fraction (e.g., SVF) 582 and polymer composition 585 are transferred from the collection vessel 110 to the delivery vessel 140. In the embodiment shown, the delivery vessel 140 comprises a plunger 146 which may be retracted to draw fluid into the delivery vessel. As retraction of the plunger 146 draws fluid into the delivery vessel 140, the polymer composition 585 and the cellular fraction (e.g., SVF) 582 mix to form a cell loaded polymer composition 590 which is ready for delivery in step 560 to the defect site for repair or regeneration of the tissue or treatment of the defect. Delivery may be arthroscopically or by open surgery.

Owing to its liquid state, the cell loaded polymer composition 590 fills the tissue defect area. Once the defect is filled, the cell loaded polymer composition 590 is activated in a step 570 to initiate an irreversible phase change from liquid to solid for in situ stabilisation of the polymer composition, thereby forming a bioscaffold, such as a hydrogel bioscaffold, for cell proliferation and tissue growth. Activation may involve initiating crosslinking of the polymer composition e.g. photocrosslinking, by exposure to light as described in the examples below.

It is to be noted that use of the apparatus 100 is not to be limited to the specifics described in connection with method 500 and variations are contemplated and expressly form part of this disclosure.

In one variation, the polymer composition 585 preloaded in the collection vessel 110 may be in a liquid state which is retained in the collection vessel by a closure, luer lock or by a valve closing the bottom opening 116 of the collection vessel, and operable by a user to control release of the contents of the collection vessel. In such an embodiment, a phase change mechanism is not required as disclosed in relation to step 540 which may simplify the process. Moreover, the cell loaded polymer composition may be prepared within the collection vessel 110 e.g. by removal of the filter barrel containing the tissue fraction to be discarded following centrifugation, and introduction of a mixing member. In some embodiments, the mixing member may comprise a plunger introduced into the collection vessel to advance the contents out through opening 118. As the polymer composition 585 and the cellular fraction (e.g., SVF) 582 are advanced out through opening 118 mixing occurs to produce the cell loaded polymer composition 590. Thus, the collection vessel 110 with a plunger (not shown) may be used to deliver the cell loaded polymer composition directly to the defect site, or to a separate delivery device 140 coupled to the opening 118.

Alternatively/additionally, the collection vessel 110 may be preloaded with a polymer composition that does not yet comprise a photoinitiator. This may be achieved by an additional step (not shown) of introducing a photoinitiator in the collection vessel before or after separation step 520. Alternatively/additionally, the photoinitiator may be introduced or preloaded in the delivery vessel 140, such that step 550 forms the cell loaded polymer composition 590, comprising a mixture of the polymer composition 585 and the cellular fraction (e.g., SVF) 582, within the delivery vessel 140. Introducing the photoinitiator to the apparatus may avoid premature crosslinking of the polymer composition (e.g. due to exposure to light during transit and storage) thereby increasing shelf life.

FIG. 11 is a schematic representation of a method 600 for preparing a treatment composition comprising a cell loaded polymer composition according to another embodiment of the disclosure. In the embodiment shown, apparatus 100 of FIGS. 1 to 2d is employed however it is to be understood that the apparatus of FIG. 3 or 5a-e could be employed as alternatives. In the embodiment shown, the apparatus is provided with the delivery vessel 140 preloaded with a volume of polymer composition 585 which is liquid at room temperature.

In a step 610, harvested tissue 580 is provided into the filter barrel 120. The tissue may be harvested arthroscopically or upon open surgery from e.g. the infrapatellar fat pad (IFP). However it is to be understood that harvested tissue may be from other regions. When tissue is harvested arthroscopically, the apparatus may be coupled with an attachment that provides for delivery of the harvested tissue from the arthroscope to the collection vessel 110 via a connected tubing system. An example of an attachment is provided in FIGS. 9a to 9c. In some embodiments, the method 600 may comprise processing the harvested tissue 580 to homogenise the sample prior to separation of the cellular fraction (e.g., SVF) 582, as described in relation to method 500 shown in FIG. 10 and the method 700 as shown in FIGS. 12a and 12b.

In a step 620, the cellular fraction (e.g., SVF) 582 is separated from the harvested tissue 580. This may be achieved by placing the apparatus 100 comprising the collection vessel 110 and the filter barrel 120 containing the harvested tissue 580 within a centrifuge (not shown) where centrifugation occurs to separate a cellular fraction (e.g., SVF) 582. The apparatus containing the harvested tissue may be centrifuged for about 3 to about 8 minutes at about 1800 to 2200 g, such as for about 5 minutes at about 2000 g using a standard laboratory centrifuge. In some embodiments, the collection vessel 110 may be configured to be received in a tube holder within the centrifuge. Alternatively, the collection vessel 110 may be configured to be received in a container such as a 50 mL centrifuge tube that is configured to be received in a tube holder within the centrifuge. At completion of separation step 620, the cellular fraction (e.g., SVF) 582 is separated out from the harvested tissue in the collection vessel 110. FIG. 11 provides a valve connector 150 coupled with collection vessel 110 to prevent loss of the cellular fraction (e.g., SVF). Alternatively, a luer lock or other closure may be provided to prevent this loss.

In a step 630, the collection vessel 110 is coupled via valve connector 150 with a delivery vessel 140. The delivery vessel 140 may be provided with a seal 142 over opening 144 to avoid loss of the preloaded polymer composition 585. Such a seal requires removal prior to coupling with valve connector 150. The valve connector 150 is opened by a user in step 640, permitting transfer of the cellular fraction (e.g., SVF) 582 into the delivery vessel 140. In the embodiment shown, the delivery vessel 140 comprises a plunger 146 which may be retracted to transfer the liquid polymer composition into the delivery vessel. With retraction of the plunger 146 the liquid polymer composition can be mixed within the delivery vessel 140. The polymer composition 585 in the delivery vessel 140 and the cellular fraction (e.g., SVF) 582 from the collection vessel 110 mix to form a cell loaded polymer composition 590 ready for delivery in step 650 to the defect site for repair or regeneration of the tissue, or the treatment of the defect. Delivery may be arthroscopically or by open surgery. Delivery may be directly through the delivery vessel opening 144, or the contents may be transferred to a delivery device, such as a biopen.

Owing to its liquid state, the cell loaded polymer composition 590 fills the tissue defect area. Once the defect is filled, the cell loaded polymer composition 590 is activated in a step 660 to initiate an irreversible phase change from liquid to solid for in situ stabilisation of the polymer composition, thereby forming a bioscaffold, such as a hydrogel bioscaffold, for cell proliferation and tissue growth. Activation may involve initiating crosslinking of the polymer e.g. photocrosslinking, by exposure to light as described in the examples below.

It is to be noted that the apparatus 100 is not to be limited to the specifics described in connection with method 600, and variations are contemplated and expressly form part of this disclosure.

In one variation, the polymer composition 585 preloaded in the delivery vessel 140 is retained by a luer lock or valve connector (not shown) in lieu of removable seal 140. This may obviate the need for valve connector 150 to be provided with the collection vessel 110 which may instead be provided with a luer lock or removable seal to prevent loss of cellular fraction (e.g., SVF) 582 through opening 118. This arrangement may provide an advantage whereby the collection vessel is more easily accommodated within the centrifuge for separation of cellular fraction (e.g., SVF) 582 in step 620. In arrangements providing a valve connector 150 between the collection vessel 110 and the delivery vessel 140, a user can control the opening between the collection vessel 110 and the delivery vessel 140 to provide further control over the rate at which the cellular fraction (e.g., SVF) 582 is released into the delivery vessel. In some embodiments, valve connector 150 may be a 3-way connector providing an access port for introduction of other liquids. In some embodiments, a 3 way connector may be used to introduce e.g. a photoinitiator (see below) or a solid to liquid phase change agent, for example, a chemical agent (e.g., a chelator such as EDTA) in examples such as FIG. 10 where the preloaded polymer composition is in a solid (e.g., gel) state.

Alternatively/additionally, the delivery vessel 140 may be preloaded with a polymer composition that does not yet comprise a photoinitiator. This may be achieved by an additional step (not shown) of introducing a photoinitiator in the delivery vessel 140 before delivery step 650, and preferably before step 640 in which the cellular fraction (e.g., SVF) is transferred into the delivery vessel so as to facilitate mixing of the photoinitiator together with the polymer composition and the cellular extract (e.g., SVF) 582. Alternatively or additionally, the photoinitiator may be introduced or preloaded in the collection vessel 110, and combined with the polymer composition when the cellular fraction (e.g., SVF) 582 and the photoinitiator are transferred to the delivery vessel 140 in step 640 thereby forming the cell loaded polymer composition 590. Introducing the photoinitiator within the apparatus may avoid degradation of the polymer composition (e.g. due to exposure to light during transit and storage) thereby increasing shelf life.

FIGS. 12a and 12b are a schematic representation of a method 700 for preparing a cell loaded polymer composition 590 according to an embodiment of the disclosure. In the embodiment shown, apparatus 100 of FIGS. 5c to 5e is employed however it is to be understood that the apparatus of FIGS. 1 to 4a-e could be employed as alternatives.

In a step 710, harvested tissue 580 is collected from a donor subject. The donor subject may be the patient being treated with the treatment composition, or a donor subject who is not the patient. The tissue may be harvested arthroscopically or upon open surgery from e.g. the infrapatellar fat pad (IFP). IFP has been proven to be a good source for adipose derived stem cells e.g. mesenchymal stem cells for cartilage regeneration application. However, it is to be understood that harvested tissue may be from other regions. When tissue is harvested arthroscopically, the arthroscopy instrument may be coupled with an attachment 900 that provides for collection of the harvested tissue from the arthroscope to e.g. a collection device 980 via a connected tubing system 990. An example of an attachment 900 is discussed with reference to FIGS. 9a-9c. The arthroscopy system provides suction and connection of the attachment 900 in the suction flow path permits collection of the harvested tissue into the attachment body 950 or a collection device 980 coupled with the attachment in a closed, sterile system. The amount of harvested tissue required for preparation of the treatment composition (e.g. 6 mL) may be drawn into a transfer container 982 (e.g. a 10 mL syringe). A connector or coupling device may be used between the collection device 980 and transfer container 982.

In some embodiments, the method 700 may comprise step 720 for processing the harvested tissue 580 to homogenise and the sample prior to separation of the cellular fraction (e.g., SVF). Processing may involve e.g. transferring the harvested tissue between 2 containers connected via e.g. a luer lock system. In some embodiments, the transfer container 982 contains the measured quantity harvested tissue and a first saline container 984 contains a sterile saline solution to assist with processing. The harvested tissue may be mixed with the sterile saline by transferring the contents between containers 982 and 984 e.g. up to 20 times, such as about 10 times or about 5 times, to achieve mixing and mincing of the tissue. In some examples the harvested tissue is combined with the sterile saline in a ratio of about 6:1.5 tissue:saline during tissue processing step 720.

In a step 730 a desired quantity (e.g. 5 mL) of the processed harvested tissue 580a, is transferred to the apparatus 100. In a step 740 a cellular fraction (e.g., SVF) 582 is separated from the processed harvested tissue 580a. This may be achieved by placing the apparatus 100, comprising the collection vessel 110 and the filter barrel 120 containing the harvested tissue 580 coupled with collection container 160, within a centrifuge tube 170 and placing in centrifuge 742. Centrifugation occurs to separate a cellular fraction (e.g., SVF) 582 into the collection container 160. The apparatus containing the harvested tissue may be centrifuged for about 3 to about 8 minutes at about 1800 to 2700 g, such as for about 5 minutes at about 2500 g using e.g. a standard laboratory centrifuge. At completion of separation step 740, the cellular fraction (e.g., SVF) 582 is separated out from the harvested tissue 580 and is collected in collection container 160. The collection container 160 may be provided with a coupling at the opening that can be releasably coupled with a closure (not shown) for storing the sterile contents not used in the immediate procedure for preparing the treatment composition.

In a step 750, the collection container 160 is coupled e.g. via a luer coupling with a mixing vessel 986 e.g. a sterile syringe of 1 mL capacity and a volume (such as about 0.05 mL) of the cellular fraction (e.g., SVF) 582 is drawn into the mixing vessel 986. In some examples, the cellular fraction in mixing vessel 986 may be mixed in a step 760 with a volume of sterile saline from a second sterile saline container 988, such as a syringe containing sterile saline. In some examples the quantity of saline mixed with the cellular fraction (e.g., SVF) 582 in mixing vessel 986 is about 0.45 mL. The mixing vessel 986 may be coupled with second sterile saline container 988 via a coupler which may have an elongated body and/or narrow bore to assist with mixing. An example is Aesthetic Group part no. FDO993922-R. The cellular fraction (e.g., SVF) 582 in mixing vessel 986 may be mixed with the sterile saline by transferring the contents between mixing vessel 986 and second sterile saline container 988 up to e.g. 20 times, such as about 10 times or about 5 times, to achieve mixing of the cells with the saline.

In a step 770, a quantity of the cell mixture 582a from step 760 is combined with the polymer composition 585. In some examples, a required quantity of the polymer composition 585 is preloaded in a polymer container 989 such as a 1 mL syringe which is capped to prevent leakage of the liquid polymer composition. Alternatively a required quantity of the polymer composition 585 may be drawn up from polymer container 989 into a syringe that is not preloaded. A desired quantity of the cell mixture 582a (e.g. 0.25 mL) may be drawn into polymer container 989 to form the cell loaded polymer composition 590. In some examples, a blending vessel 987 may be used in a step 780 to combine the cell mixture 582a with the polymer composition 585 by transferring the contents between polymer container 989 and blending vessel 987 up to e.g. 60 times, such as about 50 times or about 40 times or about 30 times, or fewer as may be the case, to achieve mixing of the cell mixture 582a with the polymer composition 585. A connector 985 which may have an elongated body and/or narrow bore may be coupled between polymer container 989 and blending vessel 987 to assist with mixing to produce the cell loaded polymer composition 585. The blending vessel 987 may comprise a 1 mL syringe and in some examples, may perform the function of the delivery vessel 140.

In a step 790, the contents of the blending vessel 987 delivery vessel 140 comprising the cell loaded polymer composition 590 may be transferred to a delivery device, such as a biopen 1730 which is ready for delivery in step 795 to the defect site for repair or regeneration of the tissue or treatment of the defect. In some examples, the polymer composition may be a liquid with photoinitiator included. Delivery may be arthroscopically or by open surgery. Owing to its liquid state, the cell loaded polymer composition 590 fills the tissue defect area. Once the defect is filled, the cell loaded polymer composition 590 is activated to initiate an irreversible phase change from liquid to solid for in situ stabilisation of the polymer composition, thereby forming a bioscaffold, such as a hydrogel bioscaffold, for cell proliferation and tissue in growth. Activation may involve initiating crosslinking of the polymer composition e.g. photocrosslinking, by exposure to light such as light having a wavelength of about 405 nm, optionally at about 20 mW/cm² for about 60 seconds to about 180 seconds, such as for about 120 seconds.

It is to be noted that the apparatus 100 and components used with the apparatus in preparation of the treatment composition is not to be limited to the specifics described in connection with method 700, and variations are contemplated and expressly form part of this disclosure.

In one variation, polymer container 989 may be preloaded with a polymer composition which is in a solid/gel state prior to mixing with the cell mixture. In such cases, a phase change step may be introduced prior to step 770 to change the state of the polymer composition 585 from solid to liquid form of lower viscosity allowing it to be mixed with the cell mixture 582a. The phase change mechanism may be selected based on the polymer composition. In one example provided the pre-loaded polymer composition 585 comprises gelatin methacryloyl (GelMA), and the phase change mechanism comprises heating the apparatus to a target temperature (typically 37 deg C.). This may be achieved by placing the polymer container 989 in an incubator, such as a standard laboratory incubator, to warm the contents. In another example provided below, the pre-loaded polymer composition comprises methacrylated alginate-RGD, and the phase change mechanism comprises addition of a solid to liquid phase change agent to the polymer composition, for example, a chemical agent (e.g., a chelator such as EDTA). It may be preferred, however that the polymer composition is provided in liquid phase to reduce the number of steps required to prepare the cell loaded polymer composition.

Alternatively or additionally, the polymer container 989 may be preloaded with a polymer composition that does not yet comprise a photoinitiator. Preparation of the polymer composition comprising the photoinitiator may be achieved by an additional step (not shown) of introducing a photoinitiator in the polymer container 989 prior to blending step 790. Alternatively or additionally, the photoinitiator may be introduced or preloaded in the blending vessel 987, such that step 790 forms the cell loaded polymer composition 590, comprising a mixture of the polymer composition 585 and the cell mixture 582a together with the photoinitiator. Introducing the photoinitiator to the apparatus may avoid premature crosslinking of the polymer composition (e.g. due to exposure to light during transit and storage) thereby increasing shelf life.

The surgical environment is complex with a wide range of monitoring equipment and instruments present to facilitate various procedures. Organisation and execution of procedures can be simplified by provision of kits that contain many or all of the consumable components required for a particular procedure, and their assembly or utilisation can follow an orderly sequence to enhance the efficiency, effectiveness and safety of the procedure. In some circumstances kits may also contain re-usable components that can be sterilised and re-used in kits prepared for further surgeries. FIGS. 13 to 16 are schematic illustrations of kits for preparation of a treatment composition for treatment or repair of damaged tissue according to embodiments of the disclosure. The kits are required to be sterile when they reach the surgical environment and are typically, therefore, manufactured as sterile sealed kits. In examples provided, items shown in broken lines designate components that may be provided as part of the kit, or which may be provided from resources outside of the kit per se.

FIG. 13 shows a kit 1700 comprising an apparatus having a collection vessel 110 preloaded with a polymer composition 585 consistent with the example shown in FIG. 10. It is to be noted that the filter element may comprise a filter barrel 120 with a strainer as shown, or it may comprise a strainer 112 attached to or formed integrally with an interior wall of the collection vessel 110 as in FIG. 3. Also provided within kit 1700 may be one or more of a delivery vessel 140, a light source 1710, a solid to liquid phase change mechanism or agent 1720 and a delivery device 1730. An attachment 900 for collecting the harvested tissue may also be provided. Components of the kit are provided in a sterile housing 1740 with a removable sterile seal (not shown). A centrifuge is necessary but need not be provided within the kit.

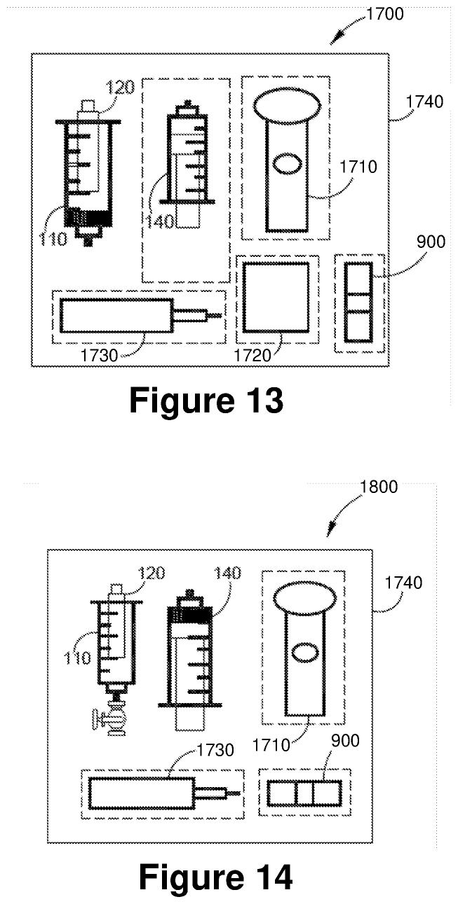

FIG. 14 shows a kit 800 comprising an apparatus 100 having a collection vessel 110, and a delivery vessel 140 preloaded with a polymer composition 585 consistent with the example shown in FIG. 11. It is to be noted that the filter element may comprise a filter barrel 120 with a strainer 112, or it may comprise a strainer 112 attached to or formed integrally with an interior wall of the collection vessel 110 as in FIG. 3. Also provided within kit 800 may be one or more of a light source 1710 and a delivery device 1730. An attachment 900 for collecting the harvested tissue may also be provided. Components of the kit are provided in a sterile housing 1740 with a removable sterile seal (not shown).

Advantageously, provision of the collection vessel preloaded with the polymer composition reduces the number of preparation or assembly steps required during the procedure. Where the polymer composition contains a photoinitiator, it is preferred that the kit housing 1740 and seal are photoopaque so as to reduce risk of premature crosslinking. Alternatively/additionally, the element containing the photoinitiator may be provided in photoopaque packaging within the kit to reduce risk of degradation.

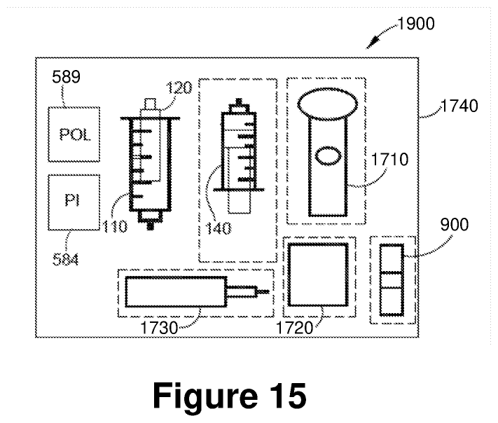

FIG. 15 shows a kit 1900 comprising an apparatus having a collection vessel 110 and a container 989 of polymer composition 585 with a separate container 584 of photoinitiator for preparation of the polymer composition within the apparatus. One or more measuring devices may be provided to measure a required amount of the polymer composition to load into the collection vessel 110. Also provided within the kit 1900 may be a delivery vessel 140, light source 1710 and a delivery device 1730. A solid to liquid phase change mechanism or agent 1720 may also be provided if the polymer is in a solid material state when the kit is sealed. An attachment 900 for collecting the harvested tissue may also be provided. Components of the kit are provided in a sterile housing 1740 with a removable sterile seal (not shown). Where the polymer composition contains a photoinitiator, it is preferred that the kit housing 1740 and seal are photoopaque so as to reduce risk of premature crosslinking, and/or that the element containing the photoinitiator may be provided in photoopaque packaging within the kit. Alternatively/additionally, the polymer composition 585 and/or the photoinitiator 584 may be provided separately from the kit 1900. This may further extend the shelf life of the kit and avoid stringent storage conditions that may require light and temperature control in order to limit degradation and/or premature crosslinking of the polymer composition 585 and/or the photoinitiator 584. A centrifuge is necessary but need not be provided within the kit.

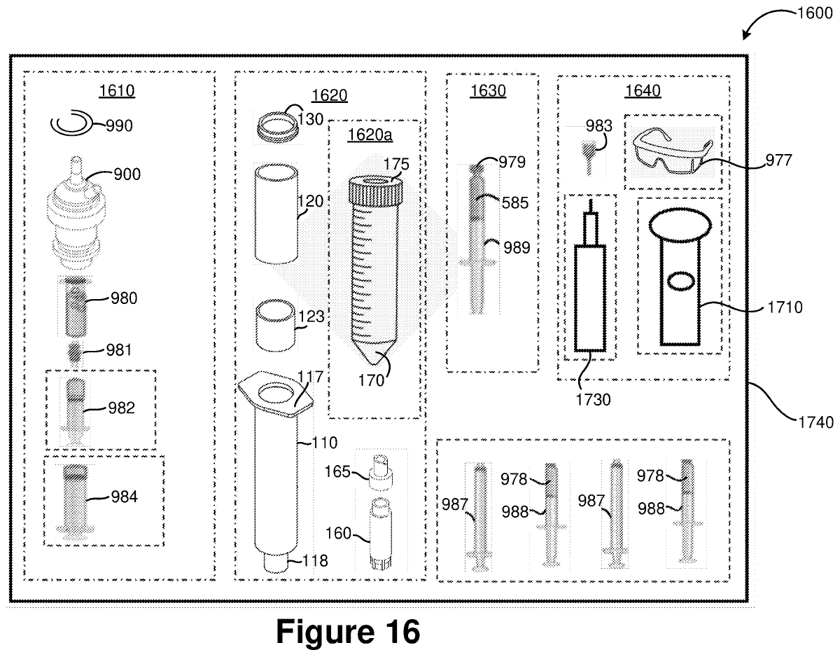

FIG. 16 shows a kit 1600 comprising items used for preparation of a treatment composition according to embodiments of the present disclosure consistent with the example described with reference to FIGS. 12a and 12b. In some examples, the kit 1600 may comprise further kits which may be individually and sterilely sealed within the kit 1600. These are referred to as kit components and are shown in dot-dash lines. The kit components may be marked with a number or name which designates the order in which the package should be opened into a sterile area for use in the preparation of the treatment composition. In other embodiments, the various kit items may be provided in a single sealed package. In other embodiments the kits represented by kit components may be supplied as individual sealed packages that are supplied separately but may be indicated for use together as disclosed herein.

One kit component may comprise a tissue collection kit 1610 comprising medical grade tubing 990, arthroscope attachment 900 and collection device 980. Coupling components for assembly of the parts of tissue collection kit 1610 may also be provided, as may transfer container 982 which may be used to draw up a desired quantity of harvested tissue, and/or a first saline container 984 the contents of which may be used to process the harvested tissue by mixing with sterile saline, such as 0.9% medical grade saline. In some examples, first saline container 984 comprises a pre-filled syringe. Alternatively, a syringe may be provided into which saline may be drawn from the sterile saline container 984 as is routinely done in the clinical setting. It is to be understood, however, that one or both of transfer container 982 and saline container 984 (and optional syringe) may be omitted from the kit 1600/1610 since these are often readily available in the clinical setting.

Another kit component may comprise apparatus kit 1620 comprising an apparatus 100 having a collection vessel 110, a filter barrel 120 comprising a strainer 121, a spacer 123, collection container 160, coupler 165 and optional stopper 130 although other apparatus configurations may be provided such as those described with reference to FIGS. 1 to 4e. A removable closure for the collection container 160 may also be provided in some cases where preservation of the cellular fraction (e.g. SVF) may be desirable. These components may be provided in the kit 1600/kit component 1620 in an assembled or unassembled form. A centrifuge tube 170 and open cap 175 may also be provided as part of the kit 1600, apparatus kit 1620, or as part of a centrifuge kit 1620a.

Another kit component may comprise polymer kit 1630 comprising a polymer container 989, such as a syringe which is prefilled with a quantity of the polymer composition. In some examples where the polymer composition contains a photoinitiator, the kit component 1630 may comprise a radio-opaque sealed bag or container to avoid or at least limit degradation prior to use. When the preloaded polymer is a liquid, the polymer container 989 may be closed with a removable stopper or seal 979 to prevent leakage in the package. In examples where the preloaded polymer is a solid or gel, a seal may not be required. It may be desirable for the polymer composition provided in the kit 1600/kit component 1630 to comprise a photoinitiator however that need not be the case and the kit 1600 or kit component 1630 may comprise a separate photoinitiator which may be mixed with the polymer composition prior to delivery of the cell-loaded polymer composition to the treatment site.

Kit 1600 or one or both of apparatus kit 1620 and polymer kit 1630 may comprise one or more blending vessels 987 and/or second sterile saline containers 988 containing saline, for preparation of a cell mixture or blending the cell loaded polymer composition as described with reference to the methods disclosed herein. The blending vessel 987 may comprise a syringe and in some examples, may perform the function of the delivery vessel 140 for providing the cell loaded polymer composition to a delivery device such as a biopen. The second sterile saline container 988 may comprise a pre-filled syringe. Alternatively, a syringe may be provided into which saline may be drawn from the second sterile saline container 988 (or the first sterile saline container 984) as is routinely done in the clinical setting. Alternatively, one or both of blending vessels 987 and second sterile saline container 988 may be omitted from the kit 1600/apparatus kit 1620/polymer kit 1630 since these are often readily available in the clinical setting.

Another kit component may comprise a delivery kit 1640 comprising an extrusion tip 983. The kit 1600/delivery kit 1640 may also comprise a delivery device such as e.g. biopen 1730. In some examples, kit 1600/delivery kit 1640 may comprise a light source 1730. The light source may be provided separately from the delivery device, or it may be e.g. integrated into the biopen. In some embodiments the kit 1600/delivery kit 1640 comprises protective eyewear 977 for use by the clinician during photoactivation of the treatment composition after delivery to the treatment site.

Components of the kit 1600 are provided in a sterile housing 1740 with a removable sterile seal (not shown), as may be the kit components. A centrifuge is necessary but need not be provided within the kit. Advantageously, provision of the apparatus in a pre-assembled form and/or provision of the polymer preloaded in the polymer container 989 reduces the number of preparation or assembly steps required during the procedure. Where the polymer composition contains a photoinitiator, it is preferred that the kit housing 1740 and seal are photo opaque so as to reduce risk of premature crosslinking. Alternatively or additionally, the element containing the photoinitiator may be provided in photo opaque packaging within the kit to reduce risk of degradation.

The following examples further illustrate aspects of the disclosure with respect to specific functional polymers.

### Example 1—Preparation of Cartilage Repair Treatment Using GelMA

Rabbit IFP tissue was retrieved and transferred into a 5 mL luer-lock sterile syringe. The retrieved IFP was minced using 2 syringes connected via a luer lock system in less than 1 minute: syringe 1=5 mL luer-lock sterile syringe containing the IFP, syringe 2=sterile syringe containing 300 ul saline solution. After mechanical processing, the syringe containing the processed IFP was coupled with the filter barrel 120 (FIGS. 1 and 2a-d) having a 40 μm strainer and the harvested tissue transferred. The collection vessel 110 was preloaded with polymer composition comprising 300 μl of GelMA 20% (Type A gelatin obtained from porcine skin, bloom 300, with about 84% methacryloyl functionalisation) with 0.2% Lithium phenyl-2,4,6 trimethylbenzoylphosphinate (LAP) (a solid/gel at room temperature).

The collection vessel 110 with filter barrel containing the processed IFP tissue was placed in a centrifuge and centrifugation took place at 2000 g for 5 minutes to purify the cellular fraction (i.e., the SVF) onto the polymer composition preloaded in the collection vessel. Following centrifugation, the collection vessel was coupled with a delivery vessel comprising a 1 mL low dead volume syringe and mounted vertically on a stage inside a 37° C.—incubator to induce a solid to liquid phase change of the polymer composition. After 5 minutes, the SVF and the polymer composition were collected into the delivery vessel 140 by drawing out a plunger from the delivery vessel to draw in and mix the fluid from the collection vessel to form the cell loaded polymer composition. Approx. 400-600 ul of cell loaded polymer composition was collected in the delivery vessel. The resulting cell loaded polymer composition was: GelMA 10% and Lithium phenyl-2,4,6 trimethylbenzoylphosphinate (LAP) 0.1% containing SVF.

The cell loaded polymer composition was delivered in vivo with open knee surgery in a rabbit animal model. Solidification of the cell loaded polymer composition was achieved via photocrosslinking with light using 405 nm light source at 20 mW/cm² for 1 minute. Samples were collected from the cartilage after 1 month from the day of treatment, where the treatment involved one of: (1) injection of native cartilage (“Cartilage”); (2) a 4 mm diameter cartilage lesion generated with a biopsy punch, that was left untreated (“Empty”); or (3) treated with the cellular fraction product mixed with a biomaterial, obtained with the apparatus (“SVF”). 8 rabbits were treated with the cell loaded polymer composition, while 4 rabbits were used for each of the Cartilage and Empty treatment groups.

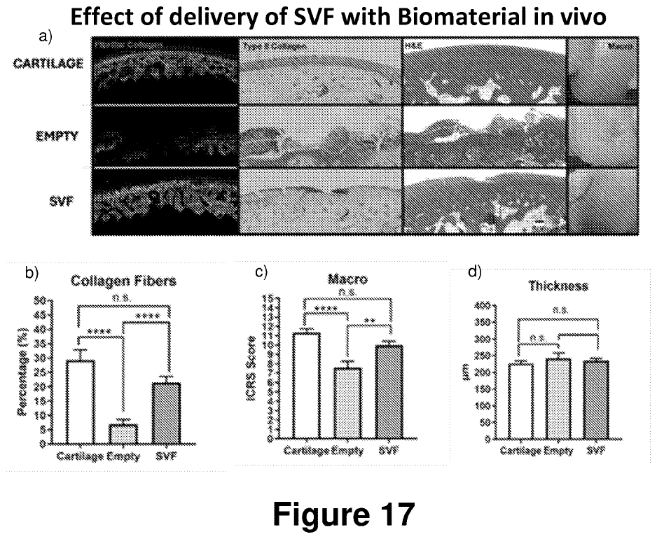

In vivo photocrosslinking to form (hydrogel) bioscaffolds was found to be compatible with cell viability, novel cartilage production and safety (FIG. 17a). Representative images of fibrillar collagen organization detected with Second Harmonic Generation microscopy (SHG), the presence of Type II Collagen in the repaired area defect, haematoxylin eosin staining (H&E) and macroscopic image of the treated areas are shown in FIG. 17. The relative amount of collagen was compared to the total region of interest (ROI) in the repaired area, and analysis performed over three ROIs for each sample; the “SVF” treated group showed no significant differences with normal cartilage (FIG. 17b). “SVF” treatment showed no significant difference in the ICRS macroscopic score and thickness of the repaired area (FIG. 17c-d), with cell loaded polymer composition treatment significantly increasing collagen fiber percentage and macroscopic score compared to Empty treatment (FIG. 17b-c).

### Example 2—Preparation of Cartilage Repair Treatment Using Methacrylated Alginate-RGD

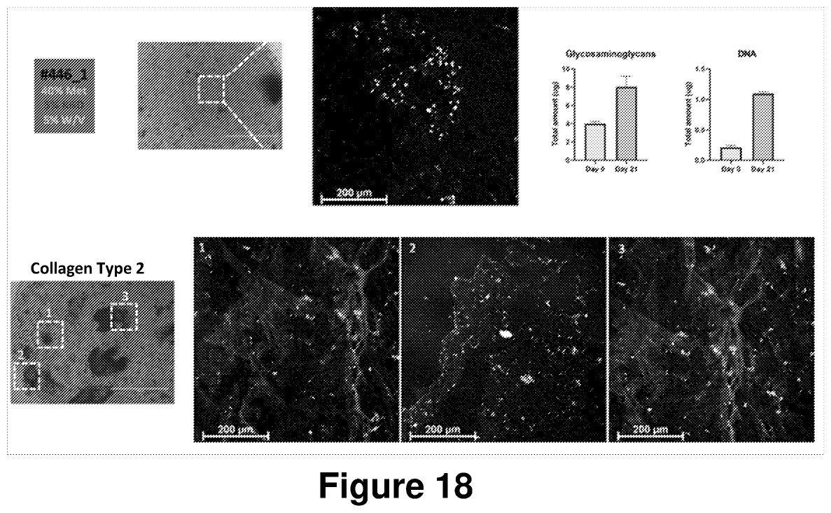

IFP tissue was retrieved and underwent mechanical processing and was transferred into filter barrel 120 as in Example 1. The delivery vessel 140 was preloaded with polymer composition comprising 400 μl of 5% methacrylated alginate-RGD (270 kDa alginate with M/G ratio of approximately 1.3, about 45-46% methacrylate functionalisation and 5-6% RGD, as a wt % substitution of reactive groups) and LAP 0.125%. The collection vessel 110 with filter barrel containing the IFP tissue was placed in a centrifuge and centrifugation took place at 2000 g for 5 minutes to purify the cellular fraction. Approx. 100 ul of cellular fraction was collected in the collection vessel 110. The collection vessel was coupled via valve connector that is opened by a user, permitting transfer of the cellular fraction into the delivery vessel. The SVF and the polymer composition were mixed within the delivery vessel 140 by drawing out a plunger from the delivery vessel to draw in and mix the components to form the cell loaded polymer composition. Approx. 400-600 ul of cell loaded polymer composition was collected in the delivery vessel. The resulting cell loaded polymer composition was: 4% methacrylated alginate-RGD and Lithium phenyl-2,4,6 trimethylbenzoylphosphinate (LAP) 0.1% containing cellular fraction (i.e., SVF). The cell loaded polymer composition was delivered in PDMS moulds. Solidification of the cell loaded polymer composition was achieved via photocrosslinking with light using 405 nm light source at 20 mW/cm² for 1 minute. The resulting scaffold was then cultivated in vitro for 21 days to check presence of Gycosaminoglycan and Collagen type 2, which indicate the formation of cartilage, and for proliferation of cells (as indicated by DNA amount) (FIG. 18).

### Example 3—IFP Retrieval with Arthroscope Connector

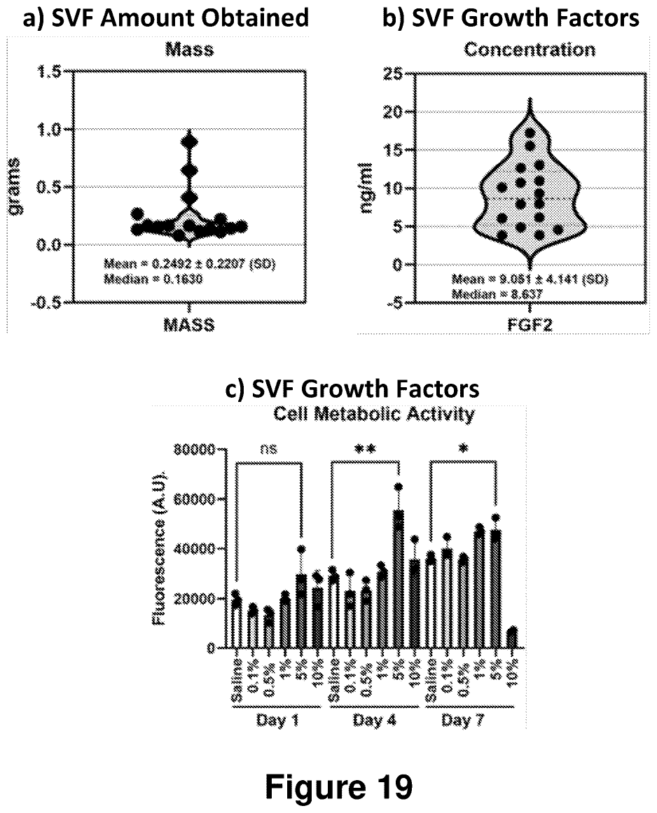

IFP tissue was retrieved and underwent mechanical processing with an arthroscope connected to the connector and was transferred into filter barrel, as described in FIG. 12a-b using a cell loaded polymer composition was: GelMA 10% and Lithium phenyl-2,4,6 trimethylbenzoylphosphinate (LAP) 0.1% containing SVF. Under this procedure 16 fat pads from 16 different patients were tested for product volume retrieval and growth factor content (FIG. 19). The mean tissue mass was 0.242 g (median value of 0.163 g) and mean fibroblast growth factor-2 (FGF-2) concentration of 9.051 ng/mL (median value of 8.637 ng/mL) (FIG. 19). Metabolic activity of human adipose derived stem cells treated with the cell loaded polymer composition at different concentrations (0.1% to 10%), was also compared to a negative control (treated with 0.9% NaCl (Saline)). The cell loaded polymer composition treatment showed a significant increase in metabolic activity of the cells when used between 1% and 5%.

### Example 4—IFP Retrieval and Cartilage Repair Treatment in Sheep Model

Cartilage repair was performed on 12 Merino sheep that received a full cartilage femoral defect of 6 mm diameter size, according to the method 700 outlined in FIGS. 12a and 12b and detailed further in this Example using the kit comprising at least the components of FIG. 16, as outlined in Table 1. In addition to the components of Table 1, the present method uses an arthroscope coupled with an attachment 900, an arthroscope shaver 5 mm full radius (sterile, individually wrapped), centrifuge (non-swing rotor with 50 mL centrifuge tube buckets), sunglasses for light exposure, 2×10 mL vials with 0.9% Medical Grade Saline, extrusion tip, and Biopen.

TABLE 1
components of the kit used in the method of Example 4

| Component | | |
|---|---|---|
| number | Component | Contents |
| 1 | Collection device | 20 mL syringe (plunger removed) |
| | 110 | Plunger from 20 mL syringe |
| | | Arthroscope attachment (2 parts- |
| | | Lid and bottom) |
| | | Combi-stopper cap |
| | | Tubing 10 cm |
| 2 | Transfer container 982 | 10 mL syringe |
| | | Luer lock transparent connector |
| | | Combi-stopper cap |
| 3 | Saline container 984 | 10 mL syringe |
| | | 0.9% Medical Grade Saline |
| | | (external to kit) |
| | | Blunt needle |
| 4 | Apparatus 100 | 10 mL collection vessel 110 |
| | | Black rubber stopper 130 |
| | | Collection capsule coupler 165 |
| | | 1.5 mL collection container 160 |
| | | 7 mL capacity strainer 121 and |
| | | spacer 123 |
| 5 | Centrifuge tube | Centrifuge open cap 175 |
| | | 50 mL Falcon tube without lid 170 |
| 6 | Mixing vessel 986 | 1 mL Low Dead Volume (low |
| | | density) Syringe |
| 7 | Saline container 988 | 1 ml syringe |
| | | Coupler |
| | | 0.9% Medical Grade Saline |
| | | (external to kit) |
| | | Blunt needle |
| 8 | Polymer container 989 | 1 mL prefilled Bioink Syringe |
| 9 | Blending vessel 987 | 1 mL Low Dead Volume Syringe |

Sheep were anaesthetised and placed in a sternal or dorsal recumbency position. A 4 cm mini arthrotomy was performed on both hind legs, involving a capsular incision and dislodging of the patellar to expose the infrapatellar fat pad (IFP). The IFP was removed from each leg, combined, and minced using the arthroscope with 5.5 mm full radius blade using the Oscillator function. The minced IFP is collected in the collection device 980, after which the arthroscope attachment was removed and discarded. The cap was then then removed from the bottom of the collection device 980, in order to connect it to the transfer container 982. 5 mL of fat was then drawn into the transfer container 982, before removing and discarding the collection device. The transfer container 982 was then connected to the saline container 984, containing 1.25 mL of saline solution, and the saline and minced IFP were mixed between the two containers 5 times back and forth.

After the final mix, the mixed fat/saline solution was located in the saline container 984, and the transfer container 982 was removed and discarded. 5 mL of the fat/saline solution was then loaded into the open end of the apparatus 100, after which the open end of the apparatus 100 was closed was a black rubber seal 130 (dome first), and the saline container 984 discarded. The apparatus 100 was placed into the centrifuge tube 170, and the combined device was centrifuged at 2500 g for 5 minutes.

During centrifugation, the chondral defect procedure was carried out to create a 6 mm biopsy punch on a weight bearing region of the condyle from both hind legs of the sheep. Within the 6 mm defect margin, the cartilage layer was removed to create a chondral defect, without opening the subchondral bone. After creating the chondral defect on both hind legs, the surgeon was then informed which leg (Left or Right) was to act as a control “Empty” defect, and which will have the cell loaded polymer composition applied.

After the centrifuge process is complete, the apparatus 100 was removed from the centrifuge tube 170, and the collection container 160, containing the cellular fraction 582, was detached from the apparatus 100. The mixing vessel 986 was used to draw up 50 ul of the composition from the collection container 160 which was then attached to the second sterile saline container 988, containing 0.450 mL of saline solution, and the two products mixed by mixing the syringes back and forth 5 times. After the final mix, the cell mixture was located in the mixing vessel 986, and the saline container 988 was removed and discarded. The polymer container 989 was then connected to the mixing vessel 986 and 250 ul of solution transferred to the polymer container 989. The blending vessel 987 was then connected to the polymer container 989 and the contents gently mixed between the two syringes 40 times. After the final mix, the mixed solution was located in the polymer container 989, the blending vessel 987 was removed and discarded, and the extrusion tip 983 was attached to the polymer container 989.

The polymer container 989 was then attached to the Biopen 1730, and the chondral lesion was filled with the cell loaded polymer composition, so that the composition was level with the outer cartilage. The Light Adaptor 1710 was then connected to the Biopen, and the light adaptor placed over the filled chondral defect. The curing light was turned on and 405 nm light source was applied to the filled chondral defect at 20 mW/cm² for 2 minutes. After photocrosslinking, the tibial plate was replaced on the femoral condyle, maintaining a gap so that the implant did not slide off the chondral lesion, and the wound closed.

Safety was constantly monitored in terms of CRS (clinical record system) scoring system, with an average CRS of 0.4 for all sheep after 4 weeks from initial treatment. The CRS being equal to 5 indicates an adverse reaction/unsafe and the CRS being equal to 0 indicates safe/no adverse reaction, and takes into account factors including food intake, drinking intake, posture, temperature, body weight loss.

### Variations on IFP

While uses of the present disclosure have been explained using the example of adipose tissue harvested from the IFP, it is to be understood that tissue harvested from other sites, particularly those tissues with cells having high chondrogenic capacity, may be suitable for use with embodiments of the novel apparatus, kits and method disclosed herein to prepare compositions for repair or regeneration of tissue, or treatment of defects in cartilage and other tissues.

As used herein “chondrogenic potential” in the context of a cell means that the cell has the capacity to promote cartilage growth, particularly hyaline cartilage. This term is applied to cells which stimulate cartilage growth, such as chondrocytes, and to cells which themselves have the capacity to differentiate into a chondrocyte under appropriate conditions. Hyaline cartilage exists on the ventral ends of ribs, in the larynx, trachea, and bronchi, and on the articulating surfaces of bones. Alternatively, harvested tissue containing cells with osteogenic and/or adipogenic potential could be used with the apparatus, kits and methods disclosed herein in the preparation of a treatment composition for the repair or regeneration of tissue, or the treatment of bone defects, osteochondral defects, cartilage defects (not only articular cartilage), or adipose tissue repair (e.g. breast reconstruction).

The mesenchymal stem cells, or related precursors, or cells derived from these cells have the capacity to form molecules of the extracellular matrix, and in particular molecules required for chondrogenesis and cartilage repair and restoration. Adipose derived stem cells (ADSCs) are particularly useful where the method is to be utilised in a procedure for cartilage repair or restoration. ADSCs may obtained from a number of different fatty tissues of the human or animal body. The ADSCs may be autologous or allogeneic.

### Advantages

The present disclosure utilises a tissue engineering approach to cartilage repair and regeneration based on highly chondrogenic stem cells in the cellular fraction (i.e., the SVF) from the IFP. The disclosure enables promotion and/or repair or regeneration of tissue such as cartilage tissue using these stem cells which may be harvested, treated and administered to the patient to achieve tissue repair, in a single sterile environment. Ideally, the harvested tissue is autologous however it is contemplated that the harvested tissue may originate from a donor source, which may be harvested in a common sterile space to the patient receiving the therapy to minimise procedure duration and risk of contamination. The novel apparatus enables the procedure to be streamlined to be achievable in a single surgical operation in an acceptable timeframe without requiring any of the patient's cells/tissues to leave the surgical theatre and without the usage of any animal derived processing enzymes.

Embodiments of the present disclosure may achieve repair or regeneration of tissue such as cartilage without the requirement to introduce additional microlesions in the bone as in the microfracture method. Furthermore, it does not require removal of healthy cartilage from a different region of the joint as in the mosaicplasty method. Embodiments of the present disclosure may achieve repair or regeneration of tissue in a single surgical procedure in a single sterile environment, and does not require isolation and expansion of cells from the donor site before the implantation, as in the ACI and MACI techniques.

Advantageously, the treatment composition of the present disclosure may be tailored for cartilage repair or regeneration due to use of a particular type of fat, already present in the knee joint, that contains cells with high chondrogenic capacity. The functionalisation of the polymer to achieve in situ stabilisation of the polymer composition fixes the cell loaded polymer composition in the defect. In contrast, lipogems use only subabdominal tissue and require additional microperforation of the lesion. Cell loaded polymer compositions prepared according to embodiments of the disclosure may contain immunomodulatory factors and also chondrogenic cells and so can enhance localised action at the repair site, representing an advantage over other repair methodologies that are deprived of any cellular component.

It is to be understood that various modifications, additions and/or alterations may be made to the parts previously described without departing from the ambit of the present invention as defined in the provisional claims appended hereto.

Future patent applications may be filed on the basis of or claiming priority from the present application. It is to be understood that the following claims are provided by way of example only, and are not intended to limit the scope of what may be claimed in any such future application. Features may be added to or omitted from the claims at a later date so as to further define or re-define the invention or inventions.

1. Apparatus for use in preparation of a composition for treatment or repair or regeneration of tissue, the apparatus comprising:
a collection vessel configured to receive harvested tissue; and
at least one filter element located inside the collection vessel;
wherein the apparatus is configured to be received in a centrifuge to separate a Stromal Vascular Fraction (SVF) from the harvested tissue within the collection vessel;
wherein at least a portion or extract of the SVF separated from the harvested tissue in the apparatus is used in preparation of a treatment composition comprising a mixture of SVF or extract and a polymer composition for treatment or repair or regeneration of the tissue.

2. The apparatus according to claim 1, comprising a collection container for receiving the separated SVF and which is removably couplable with the collection vessel.

3. The apparatus according to claim 2, wherein the collection container when coupled with the collection vessel is configured to be received within a centrifuge, or within a centrifuge tube, for use in a centrifuge.

4. The apparatus according to any one of the preceding claims, wherein the at least one filter element comprises a plurality of openings of about 30 to 60 um, preferably about 40 μm.

5. The apparatus according to any one of claims 1 to 4, wherein the filter element comprises a hollow filter barrel configured to be received within the collection vessel, and to receive the harvested tissue therein, the filter barrel comprising at least one strainer configured for separation of the SVF from the received harvested tissue.

6. The apparatus according to claim 5, wherein the apparatus comprises a spacer to separate the filter barrel from an end of the collection vessel when in use.

7. The apparatus according to any one of the preceding claims, the apparatus comprising or provided with a polymer container containing the polymer composition.

8. The apparatus according to claim 7, wherein the polymer container comprises one or both of the collection vessel and a pre-loaded syringe.

9. The apparatus according to claim 7 or claim 8, wherein the polymer container comprises a volume of liquid polymer composition, and optionally wherein the volume is about 0.5 mL to about 3 mL such as about 1 mL.

10. The apparatus according to any one of the preceding claims, the apparatus comprising or provided with a delivery vessel for receiving the prepared treatment composition, and optionally wherein the delivery vessel is couplable with a delivery device.

11. The apparatus according to any one of the preceding claims, wherein the polymer composition is functionalised to achieve in situ stabilisation of the polymer composition under activation conditions.

12. The apparatus according to claim 11, wherein the polymer composition comprises a photoinitiator and the activation conditions comprise exposure to visible light comprising a wavelength of about 405 nm, optionally at about 20 mW/cm² and optionally for about 60 seconds to about 150 seconds, preferably about 120 seconds.

13. A method for treating, repairing or regenerating tissue comprising use of the apparatus according to any one of the preceding claims.

14. A composition for treating, repairing or regenerating tissue, prepared using the apparatus of any one of claims 1 to 12 or the method of claim 13.

15. A method for repairing or regenerating tissue to treat a tissue defect comprising the steps of:
processing harvested tissue to separate a Stromal Vascular Fraction (SVF) from the harvested tissue;
preparing a treatment composition comprising a cell loaded polymer composition by mixing at least a portion of the SVF or an extract thereof with a polymer composition;
administering the treatment composition comprising the cell loaded polymer composition to the tissue defect; and
activating the cell loaded polymer composition for in situ stabilisation of the polymer composition to allow for cell ingrowth and tissue repair or regeneration;
wherein the method is performed in a single sterile environment.

16. The method of claim 15, comprising the step of obtaining the harvested tissue from a donor subject in the single sterile environment, and optionally wherein the donor subject is a patient having the tissue defect to be treated.

17. The method according to 15 or claim 16, wherein processing the harvested tissue comprises using at least one filter while centrifuging to separate the SVF.

18. The method according to any one of claims 16 to 17, comprising collecting harvested tissue using an attachment connected in a suction path of an arthroscope instrument used to remove the harvested tissue from the donor subject.

19. The method according to any one of claims 15 to 18, wherein mixing comprises, at least, transferring at least a portion the separated SVF or extract thereof and a volume of the polymer composition into a delivery vessel where the SVF portion or extract thereof, in mixture with the polymer composition, forms the cell loaded polymer composition.

20. The method according to any one of claims 15 to 19, wherein mixing comprises combining a volume of the separated SVF or extract thereof with a buffer solution before mixing with the polymer composition.

21. The method according to claim 23, wherein mixing comprises transferring a volume of the polymer composition into the combined SVF or extract and buffer solution, or vice versa, to form the cell loaded polymer composition.

22. The method according to any one of claims 15 to 21, wherein the treatment composition comprising the cell loaded polymer composition contains a photoinitiator and the activating step comprises exposing the treatment composition comprising the cell loaded polymer composition to light.

23. The method according to claim 22, wherein the activating step comprises exposing the treatment composition comprising the cell loaded polymer composition to visible light comprising a wavelength of about 405 nm, optionally at about 20 mW/cm² and optionally for about 60 seconds to about 150 seconds, preferably about 120 seconds.

24. The apparatus according to any one of claims 1 to 12 or the method according to any one of claims 15 to 23, wherein the polymer composition comprises at least one of:
gelatin methacryloyl (GelMA); and
methacrylated alginate-RGD.

25. The apparatus according to any one of claims 1 to 12 and 24 or the method according to any one of claims 15 to 24, wherein the harvested tissue is adipose tissue, preferably infrapatellar fat tissue.

26. The apparatus according to any one of claims 1 to 12, 24 or 25 or the method according to any one of claims 15 to 25, wherein the tissue being treated or repaired is cartilage, preferably articular cartilage.

27. The method according to any one of claims 19 to 26, performed using the apparatus according to any one of claims 1 to 12, or 24 to 26.

28. A kit for preparation of a composition for treatment or repair or regeneration of tissue, the kit comprising:
the apparatus according to any one of claims 1 to 12 or 24 to 26 provided in pre-assembled or unassembled condition in a kit container.

29. The kit according to claim 28, comprising an attachment for collecting harvested tissue, the attachment configured to be connected in a suction path of an arthroscope instrument used to remove the harvested tissue from a patient.

30. The kit according to claim 28 or claim 29, comprising a light source for activating the prepared composition in vivo.

31. An attachment for collecting material removed from a surgical site, the attachment comprising:
an attachment body;
an attachment inlet couplable with an arthroscope instrument configured for removing material from the surgical site by suction; and
an attachment outlet configured for fluid communication with a vacuum pump;
wherein the attachment inlet and attachment outlet are arranged such that in use, application of a vacuum at the attachment outlet draws an airstream containing material from the surgical site into the attachment inlet, and liquid and solid material in the airstream drop into the attachment body.

32. The attachment according to claim 31, wherein the attachment inlet and the attachment outlet are provided on an upper portion of the attachment.

33. The attachment according to claim 31 or claim 32, wherein the attachment inlet comprises a channel extending inside the attachment body such that in use, the airstream from the surgical site is required to change direction before exiting the attachment through the attachment outlet.

34. The attachment according to any one of claims 31 to 33, wherein the attachment comprises an attachment lid which is removable from the attachment body.

35. The attachment according to claim 34 wherein one or both of the attachment inlet and the attachment outlet are provided through the lid.

36. The attachment according to any one of claims 31 to 35, comprising an attachment container which is removably couplable with the attachment body and into which the liquid and solid material is collected when in use.

37. An attachment for use with the apparatus according to any one of claims 1 to 12 or 24 to 26, or the method of any one of claims 13 to 27, or the kit of any one of claims 28 to 30.
